(19) United States
(12) Patent Application Publication
(10) Pub. No.: US 2026/0295027 A1
(43) Pub. Date: Oct. 1, 2026

(54) ORALLY-DELIVERED VACCINES FOR DOMESTICATED AND WILD ANIMALS TO PREVENT PATHOGEN SPREAD USING RECOMBINANT PROBIOTIC BACTERIA

(71) Applicant: Colorado State University Research Foundation, Fort Collins, CO (US)

(72) Inventors: Brian D. FOY, Fort Collins, CO (US); Allison VILANDER, Fort Collins, CO (US); Gregg A. DEAN, Fort Collins, CO (US)

(21) Appl. No.: 19/345,515

(22) Filed: Sep. 30, 2025

Related U.S. Application Data
(60) Provisional application No. 63/701,172, filed on Sep. 30, 2024.

(51) Int. Cl. A61K 39/12; A61K 9/00; A61K 9/19; A61K 39/00; A61P 31/14; C07K 14/005; C12N 1/20; C12N 7/00; C12N 15/74; C12R 1/23

(52) U.S. Cl. CPC A61K 39/12; A61K 9/0056; A61K 9/19; A61P 31/14; C07K 14/005; C12N 1/20; C12N 7/00; C12N 15/746; A61K 2039/523; A61K 2039/542; A61K 2039/552; A61K 2039/55516; A61K 2039/575; C12N 2770/24022; C12N 2770/24034; C12N 2800/22; C12R 2001/23

(57) ABSTRACT

The present disclosure provides recombinant Lactobacillus acidophilus (rLA) strains comprising one or more antigens integrated into a Lactobacillus acidophilus strain. Various compositions and methods utilizing the recombinant Lactobacillus acidophilus strains are also provided, including methods of immunizing an avian to provide an immunogenic effect in the avian against West Nile Virus.

### CROSS-REFERENCE TO RELATED APPLICATIONS

This application claims the benefit under 35 U.S.C. § 119(e) of U.S. Provisional Application Ser. No. 63/701,172, filed on Sep. 30, 2024, the entire disclosures of which is incorporated herein by reference.

### GOVERNMENT RIGHTS

This invention was made with government support under grant R01 AI148633 awarded by the National Institutes of Health. The government has certain rights in the invention.

### REFERENCE TO SEQUENCE LISTING SUBMITTED ELECTRONICALLY

Incorporated by reference in its entirety is a computer-readable nucleotide/amino acid sequence listing submitted concurrently herewith and identified as follows: an XML file named “430619SequenceListing.xml”, created on Sep. 25, 2025, with a size of 34,600 bytes.

### BACKGROUND AND SUMMARY

West Nile virus (WNV) is the leading cause of domestically acquired mosquito-borne disease in the United States (U.S.), resulting in significant disease and death in humans and animals every year. Outside of the U.S., WNV is considered one of the most widespread flaviviruses in the world. From the introduction of WNV to the U.S. in 1999 through 2023, over 50,000 WNV disease cases and over 2,500 WNV-related deaths have been reported to the U.S. Center for Disease Control, and the actual number of infections is estimated to be over 5 million people. WNV has also impacted the health of North American bird populations, inducing significant population declines in several species.

The enzootic cycle of WNV is primarily maintained between Culex mosquitoes and wild passerine hosts. Infected local bridging or epidemic vectors with anthropophagic behaviors (e.g., Cx. tarsalis in the western U.S.) transmit WNV to humans and large vertebrates, typically in the latter part of the summer months.

The development and marketing of a WNV vaccine for human use remains unlikely due to the associated costs, epidemiological factors, and difficulties in planning and conducting clinical trials to establish efficacy. As a result, insecticides remain the current strategy for controlling WNV transmission. Aerially sprayed adulticides can successfully reduce WNV spread in areas throughout the U.S., but these programs are expensive. Further, success rates vary by local infrastructure, often limiting this option to wealthier urban and suburban communities. Additionally, there is limited efficacy of insecticides as a control measure due to increasing resistance in mosquitoes, off-target effects, and potential of pesticide toxicity to humans and/or other vertebrates which leads to negative community perception and decreased use. Thus, there exists a need for an alternative approach for transmission control of WNV in the ecosystem.

A strategy for immunizing avians has distinct advantages over endectocides for controlling WNV transmission, including potentially offering improved health in vaccinated both domestic and wild bird populations. Wildlife vaccine strategies have proven successful in several contexts, for instance a reduction in the spread of rabies among raccoons and other animals using a recombinant vaccina virus expressing rabies virus glycoprotein V-RG (RABORAL V-RG) in a liquid bait as well as the Borrelia burgdorferi vaccines targeted to wild white-footed mice reservoirs. However, liquid oral vaccine formulations like RABORAL V-RG are unlikely to be successful in treating wild birds.

Accordingly, the present disclosure provides recombinant Lactobacillus acidophilus (rLA) strains comprising West Nile virus protein antigens and adjuvants. The recombinant L. acidophilus strains of the present disclosure can be formulated as a lyophilized product or as part of an immunogenic composition comprising a food. Additionally, provided herein is a method of immunizing an avian by administering a recombinant L. acidophilus strain to the avian.

The recombinant L. acidophilus strains of the present disclosure provide several benefits compared to the current state of the art. For example, lactic acid bacteria, including L. acidophilus, have an easily tractable genetic system for facile heterologous gene expression in the bacilli, robust bacterial growth and protein production in cultures. Further, the simplicity and safety of non-invasive administration via oral or intranasal routes and their ability to elicit strong systemic and mucosal immunity in the treated host against foreign antigens expressed by the bacilli while eliciting minimal immunity against themselves are distinct advantages.

Additionally, the recombinant L. acidophilus strains of the present disclosure and applications thereof provide additional benefits over other strategies for controlling the spread of WNV. The use of the recombinant probiotic bacteria as a mosquito control strategy for WNV circumvents the use of insecticides and endectocides of which mosquitoes develop resistance hindering efficacy. In addition, recombinant L. acidophilus strains of the present disclosure represent a mechanism of preventing WNV over another frequently cited tool against WNV, the synthetic biology of mosquitos. Genetic modification of mosquitoes has several significant drawbacks that prevent implementation. Notably, synthetic biology-based mosquito control strategies suppress a single species, which may open an ecological niche for other species, resulting in new ecological issues. Further, for population modification approaches, it is possible that the effects designed to reduce pathogen transmission become ineffective, or force pathogen selection/evolution which may accelerate the problem. In addition, similar to the use of insecticides and endectocides, mosquito populations have a potential to evolve resistance mechanisms away from the genetic modifications.

The recombinant lactic acid bacterial strains, compositions, and methods of use present disclosure are also able to immunize wild bird populations in freedom, allowing for collective immunity. For instance, when the strains are included in an immunogenic composition, birds can ingest the immunogenic compositions and the strains become part of the bird's commensal bacterial population. This allows for continued immunization even when the wild birds are outside of the WNV endemic geographical area.

In addition, the recombinant probiotics, compositions, and methods of use provide a cost-effective means of immunizing wild bird populations. Normal vaccination campaigns would target trapping, immunizing, and releasing of wild animals, all of which take significant training, resource, and time to implement. In contrast, the present disclosure provides immunization of wild bird populations using feed, which can be placed in a given environment for self-administration by birds. In addition, the bird feed can be left for periods of time, immunizing a wider population compared to a trap-and-release campaign.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTIONS OF THE DRAWINGS

The detailed description particularly refers to the accompanying figures in which:

FIGS. 1A-1B show West Nile virus (WNV) antigen premembrane protein (prM) and adjuvant dendritic cell peptide (DCpep) can be integrated into Lactobacillus acidophilus (LA) with high fidelity. FIG. 1A depicts a graphical representation of the cloning workflow for the experiments to generate the recombinant LA (rLA) constructs. FIG. 1B shows Sanger sequencing screening of a PCR-positive colony indicating no rearrangements.

FIGS. 2A-2B show the validation of surface expression of DCpep on the rLA constructs using fluorescence-activated cell sorting (FACS). LA cells were stained with α-DCpep antibody and sorted. Wild type LA histograph of the bacterial cells is shown on the left in both FIG. 2A and FIG. 2B, while the rLA cells expressing DCpep are shown on the right. The non-overlapping plots show most cells from both constructs express DCpep on the cell surface. Majority of rLA construct populations maintain expression of adjuvant protein on cell surface. 98.1% of rLA-DCpep express DCpep on the cell surface as shown in FIG. 2A and in FIG. 2B, the population expressing DCpep is about 98.4% for the rLA-DCpep-prM cells. The results of the figure indicate that the rLA constructs continue to express the adjuvant molecule DCpep on cell surface even after multiple passages, confirming genomic maintenance and expression of integrated antigen sequence.

FIG. 3 depicts a Western blot of rLA constructs expressing integrated adjuvant and antigen proteins. Bands indicate expression of DCpep in rLA and expression of prM in construct rLA-WNV+DCpep+prM. DCpep is integrated in Surface Layer Protein A (SlpA) and expressed on the cell surface while prM is produced and maintained within the cytosol.

FIG. 4 shows a Western blot of cultured rLA constructs taken during two different growth phases (stationary and exponential). The cells were pelleted, lysed, run on an SDS-PAGE, transferred to a membrane, and the membrane stained using an α-DCpep antibody. The figure illustrates that SlpA-DCpep, where DCpep was integrated into SlpA, (which has a molecular weight of 46 kDa) is expressed in both constructs (rLA-DCpep and rLA-DCpep-prM) as expected but has higher expression in the exponential phase. Additionally, the figure shows that prM-DCpep (which has a molecular weight of 12 kDa) is observed in exponential phase lysates of rLA-DCpep-prM. This data demonstrates that prM is present in the cytosol of the rLA constructs in addition to DCpep in the SlpA protein of rLA-DCpep-prM construct.

FIGS. 5A-5C show the colonies of wild-type LA and recombinant LA (rLA). FIG. 5A depicts non-lyophilized wildtype bacteria spiral plated. The bacteria were plated on a logarithmic scale at a dilution factor of 10 to assess cell counts and viability prior to lyophilization. The plates and growth were read using a spiral plate reader giving a cell count of 4×10⁴ CFU/mL. FIG. 5B depicts lyophilized wildtype bacteria plated in the same manner at a dilution factor of 10 to assess changes in growth following lyophilization. Surprisingly, the lyophilized wildtype LA showed even greater growth than the bacteria not subjected to lyophilization, with a cell count of 8×10⁷ CFU/mL. FIG. 5C shows a light microscopy image of the lyophilized wildtype LA bacteria imaged under an oil-immersion lens. FIG. 5C further highlights the high cell counts of the bacteria encased in the protective lyophilization medium consisting of a sugar and anti-oxidant solution.

FIGS. 6A-6D show Scanning Electron Micrographs (SEM) of lyophilized rLA bound to millet. FIG. 6A and FIG. 6B show lyophilized rLA mixed with PBS and sprayed onto sterilized millet and allowed to dry. Note the rLA bacterial rods on the surface of the seeds. FIG. 6C and FIG. 6D show a SEM image following overcoating of the lyophilized rLA bound millet overcoated with a mixture of polyvinyl alcohol and apple pectin. FIG. 6A and FIG. 6C show the millet grains cut and cut viewed on the cut edge. FIG. 6B and FIG. 6D show an overhead view of the surface of the millet with lyophilized rLA bound.

FIGS. 7A-7B depicts assessments of B-cells from chicken spleens following immunization of the birds using one of the rLA constructs. The results suggest a significant increase in splenocytes secreting prM-specific IgY from oral rLA-WNV-prM culture immunization of chickens. FIG. 7A shows representative ELISpot wells. Spots indicate total and prM-specific antibody (IgY) production by incubated B-cells isolated from the birds. FIG. 7B, shows spot forming units (SFU) per million cells analyzed by ELISpot indicate similar amounts of total splenic B cells in all groups with increased prM-specific antibody response by birds treated with the rLA construct and commercial WNV vaccine (rLA-DCpep-prM and WEST NILE-INNOVATOR, WNV killed virus vaccine, respectively). Data was statistically analyzed.

### DETAILED DESCRIPTION

Various embodiments of the invention are described herein as follows. In an illustrative aspect, a recombinant Lactobacillus acidophilus (rLA) strain is provided. The recombinant Lactobacillus acidophilus (rLA) strain comprises one or more antigens integrated into a Lactobacillus acidophilus strain.

In an embodiment, the one or more antigens comprises a West Nile Virus protein. In an embodiment, the West Nile Virus protein is selected from the group consisting of a premembrane (prM) protein, an envelope protein, a non-structural protein 1 (ns1), and any combination thereof. In an embodiment, the West Nile Virus protein comprises a premembrane (prM) protein. In an embodiment, the West Nile Virus protein comprises an envelope protein. In an embodiment, the West Nile Virus protein comprises a non-structural protein 1 (ns1).

In an embodiment, the West Nile Virus protein comprises a sequence having 95% sequence identity to a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof. In an embodiment, the West Nile Virus protein comprises a sequence having 96% sequence identity to a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof. In an embodiment, the West Nile Virus protein comprises a sequence having 97% sequence identity to a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof. In an embodiment, the West Nile Virus protein comprises a sequence having 98% sequence identity to a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof. In an embodiment, the West Nile Virus protein comprises a sequence having 99% sequence identity to a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof.

In an embodiment, the West Nile Virus protein comprises a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof.

In any of the embodiments in which the West Nile Virus protein comprises a sequence, the given protein can alternatively consist essentially of the sequence or consist of the sequence.

In an embodiment, the West Nile Virus protein is encoded by a nucleic acid sequence. In an embodiment, the nucleic acid sequence is a codon optimized nucleic acid sequence.

In an embodiment, the codon optimized nucleic acid sequence comprises a nucleic acid sequence comprising 95% sequence identity to a sequence selected from the group consisting of SEQ ID NO: 2, SEQ ID NO: 4, and SEQ ID NO: 6, and a combination thereof. In an embodiment, the codon optimized nucleic acid sequence comprises a nucleic acid sequence comprising 96% sequence identity to a sequence selected from the group consisting of SEQ ID NO: 2, SEQ ID NO: 4, and SEQ ID NO: 6, and a combination thereof. In an embodiment, the codon optimized nucleic acid sequence comprises a nucleic acid sequence comprising 97% sequence identity to a sequence selected from the group consisting of SEQ ID NO: 2, SEQ ID NO: 4, and SEQ ID NO: 6, and a combination thereof. In an embodiment, the codon optimized nucleic acid sequence comprises a nucleic acid sequence comprising 98% sequence identity to a sequence selected from the group consisting of SEQ ID NO: 2, SEQ ID NO: 4, and SEQ ID NO: 6, and a combination thereof. In an embodiment, the codon optimized nucleic acid sequence comprises a nucleic acid sequence comprising 99% sequence identity to a sequence selected from the group consisting of SEQ ID NO: 2, SEQ ID NO: 4, and SEQ ID NO: 6, and a combination thereof.

In an embodiment, the codon optimized nucleic acid sequence comprises a nucleic acid sequence selected from the group consisting of SEQ ID NO: 2, SEQ ID NO: 4, and SEQ ID NO: 6, and a combination thereof.

In any of the embodiments in which the West Nile Virus protein is encoded by a nucleic acid sequence that comprises a sequence, the given protein can alternatively consist essentially of the sequence or consist of the sequence.

In an embodiment, the recombinant L. acidophilus comprises an enolase gene, and wherein the one or more antigens are integrated downstream of the enolase gene in a chromosome of the recombinant L. acidophilus strain. In an embodiment, the recombinant L. acidophilus comprises cytosol, and wherein the one or more antigens are expressed in the cytosol of the recombinant L. acidophilus strain.

In an embodiment, the recombinant L. acidophilus strain is AF196835. In an embodiment, the recombinant L. acidophilus strain is microencapsulated.

In an embodiment, the recombinant L. acidophilus strain further comprises an adjuvant. In an embodiment, the adjuvant is a dendritic cell (DC) peptide. In an embodiment, the dendritic cell (DC) peptide comprises a sequence comprising 95% sequence identity to SEQ ID NO: 14. In an embodiment, the dendritic cell (DC) peptide comprises a sequence comprising 96% sequence identity to SEQ ID NO: 14. In an embodiment, the dendritic cell (DC) peptide comprises a sequence comprising 97% sequence identity to SEQ ID NO: 14. In an embodiment, the dendritic cell (DC) peptide comprises a sequence comprising 98% sequence identity to SEQ ID NO: 14. In an embodiment, the dendritic cell (DC) peptide comprises a sequence comprising 99% sequence identity to SEQ ID NO: 14.

In an embodiment, the dendritic cell (DC) peptide comprises SEQ ID NO: 14.

In any of the embodiments in which the adjuvant comprises a sequence, the given adjuvant can alternatively consist essentially of the sequence or consist of the sequence.

In an embodiment, the adjuvant is encoded by a nucleic acid sequence comprising 95% sequence identity to SEQ ID NO: 13. In an embodiment, the adjuvant is encoded by a nucleic acid sequence comprising 96% sequence identity to SEQ ID NO: 13. In an embodiment, the adjuvant is encoded by a nucleic acid sequence comprising 97% sequence identity to SEQ ID NO: 13. In an embodiment, the adjuvant is encoded by a nucleic acid sequence comprising 98% sequence identity to SEQ ID NO: 13. In an embodiment, the adjuvant is encoded by a nucleic acid sequence comprising 99% sequence identity to SEQ ID NO: 13. In an embodiment, the adjuvant is encoded by a nucleic acid sequence comprising SEQ ID NO: 13. In any of the embodiments in which the adjuvant is encoded by a nucleic acid sequence that comprises a sequence, the given adjuvant can alternatively consist essentially of the sequence or consist of the sequence.

In an embodiment, the recombinant L. acidophilus strain comprises a surface layer protein A (slpA) gene, and wherein the adjuvant is integrated into the surface layer protein A (slpA) gene in the recombinant L. acidophilus strain.

In an embodiment, the recombinant L. acidophilus strain comprises a 3′ end of the nucleic acid sequence, and wherein the adjuvant is integrated at the 3′ end of any one of the nucleic acid sequences encoding one or more of the West Nile Virus proteins. In an embodiment, the adjuvant integrated at the 3′ end of any one of the nucleic acid sequences encoding one or more of the West Nile Virus proteins comprises a nucleotide sequence having 95% sequence identity to any one of SEQ ID NO: 1, SEQ ID NO: 3, SEQ ID NO: 5, and any combination thereof. In an embodiment, the adjuvant integrated at the 3′ end of any one of nucleic acid sequences encoding one or more of the West Nile Virus proteins comprises a nucleotide sequence having 96% sequence identity to any one of SEQ ID NO: 1, SEQ ID NO: 3, SEQ ID NO: 5, and any combination thereof. In an embodiment, the adjuvant integrated at the 3′ end of any one of nucleic acid sequences encoding one or more of the West Nile Virus proteins comprises a nucleotide sequence having 97% sequence identity to any one of SEQ ID NO: 1, SEQ ID NO: 3, SEQ ID NO: 5, and any combination thereof. In an embodiment, the adjuvant integrated at the 3′ end of any one of nucleic acid sequences encoding one or more of the West Nile Virus proteins comprises a nucleotide sequence having 98% sequence identity to any one of SEQ ID NO: 1, SEQ ID NO: 3, SEQ ID NO: 5, and any combination thereof. In an embodiment, the adjuvant integrated at the 3′ end of any one of nucleic acid sequences encoding one or more of the West Nile Virus proteins comprises a nucleotide sequence having 99% sequence identity to any one of SEQ ID NO: 1, SEQ ID NO: 3, SEQ ID NO: 5, and any combination thereof.

In an embodiment, the adjuvant integrated at the 3′ end of any one of nucleic acid sequences encoding one or more of the West Nile Virus proteins comprises a nucleotide sequence comprising any one of SEQ ID NO: 1, SEQ ID NO: 3, SEQ ID NO: 5, and any combination thereof.

In an illustrative aspect, a lyophilized product comprising the recombinant Lactobacillus acidophilus strain is provided. The recombinant Lactobacillus acidophilus strain can comprise any of the rLA strains described herein.

In an embodiment, the lyophilized product comprises one or more recombinant L. acidophilus strains comprising a mixture of West Nile Virus proteins.

In an illustrative aspect, an immunogenic composition comprising a food composition and the lyophilized product is provided. The lyophilized product can comprise any of the lyophilized products described herein.

In an embodiment, the lyophilized product is resuspended in a liquid. In an embodiment, the liquid is water. In an embodiment, the liquid is phosphate buffered saline (PBS).

In an embodiment, the lyophilized product is sprayed onto the feed composition.

In an embodiment, the immunogenic composition comprises the lyophilized product at between about 10⁶ to about 10¹² viable microorganisms of the food composition or between about 10⁶ to about 10¹² microorganisms of the recombinant L. acidophilus strains per gram of the food composition.

In an embodiment, the immunogenic composition comprises the lyophilized product at about 10⁶ viable microorganisms of the recombinant L. acidophilus strain per gram of the food composition. In an embodiment, the immunogenic composition comprises the lyophilized product at about 10⁷ viable microorganisms of the recombinant L. acidophilus strain per gram of the food composition. In an embodiment, the immunogenic composition comprises the lyophilized product at about 10⁸ viable microorganisms of the recombinant L. acidophilus strain per gram of the food composition. In an embodiment, the immunogenic composition comprises the lyophilized product at about 10⁹ viable microorganisms of the recombinant L. acidophilus strain per gram of the food composition. In an embodiment, the immunogenic composition comprises the lyophilized product at about 10¹⁰ viable microorganisms of the recombinant L. acidophilus strain per gram of the food composition. In an embodiment, the immunogenic composition comprises the lyophilized product at about 10¹¹ viable microorganisms of the recombinant L. acidophilus strain per gram of the food composition. In an embodiment, the immunogenic composition comprises the lyophilized product at about 10¹² viable microorganisms of the recombinant L. acidophilus strain per gram of the food composition.

In an embodiment, the immunogenic composition comprises the lyophilized product at about 5×10⁶ viable microorganisms of the recombinant L. acidophilus strain per gram of the food composition. In an embodiment, the immunogenic composition comprises the lyophilized product at about 5×10⁷ viable microorganisms of the recombinant L. acidophilus strain per gram of the food composition. In an embodiment, the immunogenic composition comprises the lyophilized product at about 5×10⁸ viable microorganisms of the recombinant L. acidophilus strain per gram of the food composition. In an embodiment, the immunogenic composition comprises the lyophilized product at about 5×10⁹ viable microorganisms of the recombinant L. acidophilus strain per gram of the food composition. In an embodiment, the immunogenic composition comprises the lyophilized product at about 5×10¹⁰ viable microorganisms of the recombinant L. acidophilus strain per gram of the food composition. In an embodiment, the immunogenic composition comprises the lyophilized product at about 5×10¹¹ viable microorganisms of the recombinant L. acidophilus strain per gram of the food composition. In an embodiment, the immunogenic composition comprises the lyophilized product at about 5×10¹² viable microorganisms of the recombinant L. acidophilus strain per gram of the food composition.

In an embodiment, the food composition further comprises a protective material. In an embodiment, the protective material comprises a polyvinyl alcohol. In an embodiment, the protective material comprises a polysaccharide. In an embodiment, the polysaccharide is at a concentration of 10% w/v. In an embodiment, the polysaccharide is selected from the group consisting of pectin, sodium alginate, insulin, 6-glucans, dextran, agar, carrageenans, xanthan, maltodextrins, and a combination thereof.

In an embodiment, the protective material comprises a combination of a polyvinyl alcohol and a polysaccharide. In an embodiment, the combination is present at a 4:1 ratio of the polyvinyl alcohol to the polysaccharide.

In an embodiment, the food composition comprises a bird feed. In an embodiment, the bird feed is selected from the group consisting of sunflower, safflower, nyjer or thistle, millet, corn, peanuts, milo or sorghum, flax, rapeseed (Brassica napus), canary seed (Phalaris canariensis), fruit, suet, mealworms, and a combination thereof.

In an embodiment, the bird feed is a millet. In an embodiment, the millet is selected from the group consisting of golden millet, red millet, white proso millet, and a combination thereof.

In an embodiment, the bird feed is a fruit. In an embodiment, the fruit is fresh. In an embodiment, the fruit is dried. In an embodiment, the fruit is selected from the group consisting of cranberries, cherries, grapes, apples, oranges, melons, and a combination thereof.

In an embodiment, the bird feed is hulled. In an embodiment, the bird feed is unhulled.

In an embodiment, the immunogenic composition further comprises a functional ingredient selected from the group consisting of a trace element, a mineral, a vitamin, an antioxidant, a sterol, a polyunsaturated fatty acids (PUFA), an amino acids, and a combination thereof.

In an embodiment, the functional ingredient is a trace element. In an embodiment, the trace element is selected from the group consisting of boron, cobalt, chloride, chromium, copper, fluoride, iodine, iron, manganese, molybdenum, selenium, zinc, and a combination thereof.

In an embodiment, the functional ingredient is a mineral. In an embodiment, the mineral is selected from the group consisting of calcium, magnesium, potassium, sodium, and a combination thereof.

In an embodiment, the functional ingredient is a vitamin. In an embodiment, the vitamin is selected from the group consisting of vitamin A, vitamin D3, vitamin E, vitamin K, vitamin B12, biotin, choline, vitamin B1, vitamin B2, vitamin B6, niacin, folic acid, panthothenate, and a combination thereof.

In an embodiment, the functional ingredient is an antioxidant. In an embodiment, the antioxidant is selected from the group consisting of carotenoids, lycopene, beta-carotene, lutein, xanthophylls, vitamin A, tocopherols, vitamin C, and a combination thereof.

In an embodiment, the functional ingredient is a sterol. In an embodiment, the sterol is selected from the group consisting of phytosterols, β-sitosterol, β-sitostanol, campestanol, campesterol, stigmasterol, and a combination thereof.

In an embodiment, the functional ingredient is a polyunsaturated fatty acid (PUFA). In an embodiment, the polyunsaturated fatty acid (PUFA) is selected from the group consisting of C18, C20 polyunsaturated fatty acids, C22 polyunsaturated fatty acids, and a combination thereof.

In an embodiment, the functional ingredient is an amino acid. In an embodiment, the amino acid is selected from the group consisting of lysine, alanine, beta-alanine, threonine, methionine, tryptophan, and a combination thereof.

In an illustrative aspect, a method of immunizing an avian is provided. The method comprises a step of administering a recombinant L. acidophilus strain to the avian, wherein the method provides an immunogenic effect in the avian.

In an embodiment, the immunogenic effect comprises stimulation of antigen specific immunity to a West Nile Virus protein. In an embodiment, the immunogenic effect comprises a reduction in West Nile Virus titer in the avian.

In an embodiment, the recombinant L. acidophilus strain is comprised in a lyophilized product. In an embodiment, the recombinant L. acidophilus strain is comprised in a feed composition.

In an embodiment, the avian is selected from the group consisting of a native avian, a native-captive avian, an introduced avian, and an exotic-captive avian.

In an embodiment, the avian is a native avian. In an embodiment, the native avian is selected from the group consisting of Acorn Woodpecker, American Coot, American Crow, American Dipper, American Goldfinch, American Kestrel, American Robin, American White Pelican, Anna's Hummingbird, Ash-throated Flycatcher, Bald Eagle, Baltimore Oriole, Band-tailed Pigeon, Bank Swallow, Barn Owl, Barn Swallow, Barred Owl, Belted Kingfisher, Black Phoebe, Black Skimmer, Black Vulture, Black-billed Magpie, Black-capped Chickadee, Black-chinned Hummingbird, Black-chinned Sparrow, Black-crowned Night Heron, Black-headed Grosbeak, Blackpoll Warbler, Black-throated Blue Warbler, Black-throated Gray Warbler, Black-whiskered Vireo, Blue Jay, Boat-tailed Grackle, Bobolink, Brewer's Blackbird, Broad-winged Hawk, Bronzed Cowbird, Brown Thrasher, Brown-headed Cowbird, Bullock's Oriole, Burrowing Owl, Bushtit, Cackling Goose, Cactus Wren, California Condor, California Gull, California Quail, California Towhee, Canada Goose, Canada Warbler, Carolina Chickadee, Carolina Wren, Caspian Tern, Cassin's Finch, Cattle Egret, Cedar Waxwing, Chestnut-backed Chickadee, Chihuahuan Raven, Chimney Swift, Chipping Sparrow, Cinnamon Teal, Clapper Rail, Clark's Grebe, Clark's Nutcracker, Cliff Swallow, Common Black-Hawk, Common Grackle, Common Ground-Dove, Common Loon, Common Moorhen, Common Murre, Common Nighthawk, Common Raven, Common Yellowthroat, Cooper's Hawk, Costa's Hummingbird, Dark-eyed Junco, Dickcissel, Double-crested Cormorant, Downy Woodpecker, Eastern Bluebird, Eastern Kingbird, Eastern Phoebe, Eastern Screech-Owl, Eastern Towhee, Elf Owl, Eurasian Wigeon, Evening Grosbeak, Ferruginous Hawk, Field Sparrow, Fish Crow, Flammulated Owl, Fox Sparrow, Gila Woodpecker, Glaucous-winged Gull, Golden Eagle, Golden-crowned Sparrow, Gray Catbird, Gray-cheeked Thrush, Great Black-backed Gull, Great Blue Heron, Great Crested Flycatcher, Great Egret, Great Horned Owl, Greater Prairie-Chicken, Greater Roadrunner, Greater Sage-Grouse, Greater Scaup, Greater White-fronted Goose, Great-tailed Grackle, Green Heron, Hairy Woodpecker, Hammond's Flycatcher, Harris' Hawk, Hermit Thrush, Herring Gull, Hooded Merganser, Hooded Oriole, Hooded Warbler, House Finch, House Wren, Inca Dove, Kentucky Warbler, Killdeer, Lark Sparrow, Laughing Gull, Lazuli Bunting, Least Bittern, Least Tern, LeConte's Thrasher, Lesser Goldfinch, Lesser Nighthawk, Lesser Scaup, Lewis' Woodpecker, Limpkin, Lincoln's Sparrow, Loggerhead Shrike, Long-eared Owl, MacGillivray's Warbler, Mallard, Merlin, Mexican Jay, Mississippi Kite, Mottled Duck, Mountain Bluebird, Mountain Chickadee, Mountain Quail, Mourning Dove, Nashville Warbler, Northern Bobwhite, Northern Cardinal, Northern Flicker, Northern Goshawk, Northern Harrier, Northern Mockingbird, Northern Parula, Northern Saw-whet Owl, Northern Waterthrush, Nuttall's Woodpecker, Oak Titmouse, Olive-sided Flycatcher, Orange-crowned Warbler, Orchard Oriole, Osprey, Ovenbird, Pacific-slope Flycatcher, Pelagic Cormorant, Peregrine Falcon, Pied-billed Grebe, Pine Siskin, Pinyon Jay, Piping Plover, Prairie Falcon, Purple Finch, Purple Gallinule, Purple Martin, Pygmy Nuthatch, Red Crossbill, Red-bellied Woodpecker, Red-breasted Nuthatch, Red-breasted Sapsucker, Red-eyed Vireo, Red-headed Woodpecker, Red-shouldered Hawk, Red-tailed Hawk, Red-winged Blackbird, Ring-billed Gull, Rock Wren, Rose-breasted Grosbeak, Rough-legged Hawk, Ruby-throated Hummingbird, Ruddy Duck, Ruddy Turnstone, Ruffed Grouse, Rufous Hummingbird, Rusty Blackbird, Sandhill Crane, Savannah Sparrow, Scarlet Tanager, Scissor-tailed Flycatcher, Sharp-shinned Hawk, Short-eared Owl, Snowy Egret, Song Sparrow, Sora, Spotted Towhee, Steller's Jay, Swainson's Hawk, Swainson's Thrush, Swallow-tailed Kite, Swamp Sparrow, Tennessee Warbler, Thayer's Gull, Townsend's Warbler, Tree Swallow, Tricolored Blackbird, Tufted Titmouse, Tundra Swan, Turkey Vulture, Varied Thrush, Veery, Virginia Rail, Warbling Vireo, Western Bluebird, Western Kingbird, Western Meadowlark, Western Sandpiper, Western Screech-Owl, Western Scrub-Jay, Western Tanager, Whip-poor-will, White-breasted Nuthatch, White-crowned Pigeon, White-crowned Sparrow, White-faced Ibis, White-tailed Kite, White-winged Dove, Wild Turkey, Willow Flycatcher, Wilson's Warbler, Winter Wren, Wood Thrush, Yellow Warbler, Yellow-bellied Sapsucker, Yellow-billed Cuckoo, Yellow-billed Magpie, Yellow-crowned Night-Heron, Yellow-rumped Warbler, and Yellow-throated Warbler.

In an embodiment, the avian is a native-captive avian. In an embodiment, the native-captive avian is selected from the group consisting of Boreal Owl, Bufflehead, Canvasback, Common Goldeneye, Common Merganser, Emperor Goose, Great Gray Owl, Green-winged Teal, Gyrfalcon, Muscovy Duck, Northern Hawk-Owl, Northern Pintail, Snow Goose, Snowy Owl, Spotted Owl, Whooping Crane, and Wood Duck.

In an embodiment, the avian is an introduced avian. In an embodiment, the introduced avian is selected from the group consisting of Chukar, Eurasian Collared-Dove, European Starling, House Sparrow, Mute Swan, Ringed Turtle-Dove, Ring-necked Pheasant, and Rock Pigeon.

In an embodiment, the avian is an exotic-captive avian. In an embodiment, the exotic-captive avian is selected from the group consisting of Abyssinian Ground-Hornbill, African Grey Parrot, African Penguin, Black-capped Lory, Blue-crowned Conure, Blue-eared Pheasant, Blue-streaked Lory, Blythe's Tragopan, Bronze-winged Duck, Budgerigar, Canary-winged Parakeet, Chilean Flamingo, Chinese Goose, Cinereus Vulture, Cockatiel, Cockatoo, Common Canary, Common Peafowl, Crimson-fronted Parakeet, Crimson Rosella, Domestic Chicken, Dusky Lory, Elegant Crested Tinamou, Emu, Eurasian Jay, European Goldfinch, Gouldian Finch, Greater Flamingo, Guanay Cormorant, Hawaiian Goose (Nene), Hooded Crow, Humboldt Penguin, Impeyan Pheasant, Inca Tern, Macaw, Micronesian Kingfisher, Monal Pheasant, Nutmeg Mannikin, Pacific Parrotlet, Pale-headed Rosella, Palm Tanager, Puna Teal, Rainbow Lorikeet, Red Lory, Red-breasted Goose, Red-crowned Parrot, Satyr Tragopan, Scarlet Ibis, Smew, Society Finch, Tawny Owl, Thick-billed Parrot, Varied Tit, Violet-necked Lorikeet, Wedge-tailed Eagle, Yellow-billed Duck, Zebra Finch, and Zenaida Dove.

The sequences described in the present disclosure are summarized in Tables 1-5 below.

TABLE 1
West Nile Virus nucleic acid sequences (antigens)

| SEQ ID | | | |
|---|---|---|---|
| NO | Name | Features | Sequence |
| 1 | Premembrane | start | ATGGTTACCCTCTCTAACTTCCAAGGGAAGGTGATGATGAC |
| | (prM) protein | codon, | GGTAAATGCTACTGACGTCACAGATGTCATCACGATTCCAA |
| | | stop | CAGCTGCTGGAAAGAACCTATGCATTGTCAGAGCAATGGAT |
| | | codon, | GTGGGATACATGTGCGATGATACTATCACTTATGAATGCCCA |
| | | DC | GTGCTGTCGGCTGGTAATGATCCAGAAGACATCGACTGTTG |
| | | peptide | GTGCACAAAGTCAGCAGTCTACGTCAGGTATGGAAGATGCA |
| | | | CCAAGACACGCCACTCAAGACGCAGTCGGAGGTTCTACCCA |
| | | | TCATACCACTCTACCCCACAACGCCCTTAA |
| 2 | Premembrane | codon | GTTACCCTCTCTAACTTCCAAGGGAAGGTGATGATGACGGT |
| | (prM) protein | optimized | AAATGCTACTGACGTCACAGATGTCATCACGATTCCAACAG |
| | | | CTGCTGGAAAGAACCTATGCATTGTCAGAGCAATGGATGTG |
| | | | GGATACATGTGCGATGATACTATCACTTATGAATGCCCAGTG |
| | | | CTGTCGGCTGGTAATGATCCAGAAGACATCGACTGTTGGTG |
| | | | CACAAAGTCAGCAGTCTACGTCAGGTATGGAAGATGCACCA |
| | | | AGACACGCCACTCAAGACGCAGTCGGAGG |
| 3 | Envelope (E) | start | ATGTTTAACTGTTTGGGTATGTCTAACCGTGACTTCCTTGAA |
| | | codon, | GGAGTAAGCGGTGCCACATGGGTCGATCTTGTTCTTGAGGG |
| | | stop | TGATTCTTGCGTTACCATTATGAGTAAGGACAAACCAACAAT |
| | | codon, | CGACGTCAAAATGATGAATATGGAGGCTGCCAACTTAGCTG |
| | | DC | ACGTCCGCTCATACTGTTATTTGGCTTCTGTCAGCGATCTTTC |
| | | peptide | TACCCGTGCCGCATGTCCTACAATGGGTGAGGCTCACAATG |
| | | | AGAAGCGAGCAGACCCAGCTTTTGTCTGCAAGCAGGGCGTA |
| | | | GTTGATCGAGGCTGGGGTAATGGATGTGGATTATTTGGCAA |
| | | | AGGATCTATTGACACCTGCGCAAAGTTCGCTTGCACTACCAA |
| | | | AGCCACAGGTTGGATCATTCAAAAGGAGAATATCAAATACG |
| | | | AGGTAGCCATTTTCGTACACGGACCAACAACTGTTGAGTCA |
| | | | CATGGCAAAATCGGTGCAACTCAAGCAGGCAGATTTTCTAT |
| | | | TACCCCTTCAGCACCATCATACACTTTAAAGCTTGGAGAGTA |
| | | | TGGCGAGGTTACAGTTGATTGCGAACCAAGAAGCGGAATCG |
| | | | ACACCTCAGCTTACTACGTCATGTCTGTAGGCGAGAAAAGTT |
| | | | TTTTAGTACACAGAGAGTGGTTCATGGATCTTAATTTGCCTT |
| | | | GGAGTTCTGCCGGCTCTACCACATGGCGCAATCGCGAAACC |
| | | | CTTATGGAGTTCGAGGAGCCTCATGCCACCAAGCAATCTGTT |
| | | | GTTGCCTTGGGTAGTCAAGAAGGTGCCTTGCATCAGGCACTT |
| | | | GCTGGCGCTATTCCTGTTGAGTTTAGTTCTAACACCGTCAAA |
| | | | TTGACCTCAGGTCACTTGAAATGTCGAGTTAAGATGGAAAA |
| | | | GCTTCAACTTAAGGGTACCACTTATGGCGTTTGCTCTAAGGC |
| | | | CTTTAAGTTCGCTCGTACTCCTGCCGATACCGGACATGGTAC |
| | | | CGTCGTTCTTGAGCTTCAGTACACTGGAACCGACGGTCCTTG |
| | | | TAAAGTTCCTATTAGCTCTGTCGCCTCTTTAAACGATTTGAC |
| | | | CCCTGTCGGACGTTTGGTAACAGTAAATCCATTCGTCAGTGT |
| | | | AGCAACCGCAAACTCAAAAGTATTAATTGAGTTAGAGCCTC |
| | | | CATTTGGCGACTCTTATATCGTTGTTGGACGAGGCGAACAGC |
| | | | AAATTAATCATCATTGGCACAAATCAGGAAGCAGCATCGGC |
| | | | AAGGCATTTACTACTACATTGCGTGGCGCACAAAGATTGGC |
| | | | TGCACTTGGAGATACCGCCTGGGATTTCGGCAGCGTTGGCG |
| | | | GAGTTTTCACCTCTGTAGGCAAAGCAATCCACCAAGTCTTTG |
| | | | GTGGCGCCTTCCGCAGCTTATTCGGTGGAATGTCATGGATTA |
| | | | CACAGGGATTACTTGGTGCCCTTTTGCTTTGGATGGGTATCA |
| | | | ACGCTAGAGACCGCAGCATTGCAATGACCTTTTTAGCTGTCG |
| | | | GTGGTGTATTGCTTTTTCTTTCAGTCAACGTACACGCTTTCTA |
| | | | CCCATCATACCACTCTACCCCACAACGCCCTTAA |
| 4 | Envelope (E) | codon | TTTAACTGTCTTGGCATGTCAAACCGCGATTTCCTTGAGGGA |
| | | optimized | GTAAGCGGCGCCACATGGGTCGACTTGGTCCTTGAAGGCGA |
| | | | TTCTTGCGTAACCATCATGTCTAAAGATAAGCCAACAATCGA |
| | | | TGTAAAGATGATGAACATGGAGGCCGCCAATTTAGCTGAGG |
| | | | TACGCAGTTACTGTTACCTTGCTACTGTAAGCGATTTGTCAA |
| | | | CTAAGGCCGCATGTCCAACAATGGGAGAAGCACATAACGAT |
| | | | AAGCGCGCCGACCCTGCTTTCGTATGCCGACAGGGAGTTGTT |
| | | | GATAGAGGCTGGGGAAACGGATGTGGTTTATTTGGAAAAGG |
| | | | TTCAATCGATACCTGTGCTAAGTTTGCATGTAGCACAAAAGC |
| | | | CATCGGTAGAACTATTTTGAAAGAAAACATCAAATACGAGG |
| | | | TTGCCATCTTTGTACATGGACCAACCACTGTTGAGAGCCACG |
| | | | GTAACTATTCAACACAGGTTGGCGCTACACAGGCCGGACGT |
| | | | TTCAGCATCACACCAGCAGCACCATCTTATACTTTAAAATTG |
| | | | GGAGAGTATGGCGAAGTCACCGTAGACTGTGAGCCACGATC |
| | | | TGGAATTGATACCAATGCCTATTACGTTATGACTGTTGGTAC |
| | | | CAAAACTTTTTTAGTACATCGCGAATGGTTTATGGACTTAAA |
| | | | CTTACCTTGGTCAAGCGCCGGCAGTACCGTTTGGCGAAATCG |
| | | | CGAGACCTTAATGGAGTTCGAAGAGCCACACGCCACTAAGC |
| | | | AGAGCGTCATTGCATTGGGCTCTCAGGAGGGTGCATTACAT |
| | | | CAGGCACTTGCAGGAGCCATCCCAGTAGAATTCTCAAGTAA |
| | | | TACAGTTAAGTTAACAAGTGGACATCTTAAATGTCGCGTCA |
| | | | AAATGGAGAAATTGCAGTTGAAGGGTACAACTTATGGCGTC |
| | | | TGTTCTAAGGCTTTCAAATTTTTAGGCACCCCAGCCGACACA |
| | | | GGTCACGGCACTGTCGTTCTTGAATTGCAATATACAGGAAC |
| | | | AGATGGACCTTGTAAAGTACCTATCAGCTCAGTCGCCAGTCT |
| | | | TAATGACCTTACACCTGTTGGCCGACTTGTTACTGTTAACCC |
| | | | TTTTGTCTCTGTTGCAACCGCTAACGCTAAGGTATTAATCGA |
| | | | ACTTGAGCCACCATTCGGAGACTCTTATATCGTAGTCGGTCG |
| | | | AGGAGAGCAGCAAATCAACCACCATTGGCACAAATCTGGAA |
| | | | GCAGCATCGGTAAGGCTTTTACCACTACATTAAAGGGAGCC |
| | | | CAGCGACTTGCAGCCCTTGGCGACACAGCTTGGGACTTTGG |
| | | | CTCTGTTGGAGGAGTTTTTACTTCTGTTGGAAAAGCAGTCCA |
| | | | CCAGGTTTTCGGCGGAGCCTTTAGAAGTTTATTTGGAGGTAT |
| | | | GAGTTGGATCACCCAAGGATTACTTGGAGCTTTGCTTCTTTG |
| | | | GATGGGTATTAATGCCCGAGATCGTAGTATTGCCTTAACTTT |
| | | | TCTTGCAGTAGGTGGAGTTTTATTATTTTTAAGTGTTAACGT |
| | | | CCATGCA |
| 5 | non-structural | start | ATGGACACCGGTTGCGCCATCGACATTAGTCGACAAGAATT |
| | protein 1 (ns1) | codon, | ACGTTGCGGTTCAGGAGTATTCATCCATAACGACGTAGAAG |
| | | stop | CTTGGATGGACAGATACAAGTACTATCCAGAGACCCCACAG |
| | | codon, | GGACTTGCAAAGATCATCCAAAAGGCACATAAGGAAGGAGT |
| | | DC | CTGCGGTCTTAGATCTGTCTCTCGATTGGAGCATCAGATGTG |
| | | peptide | GGAGGCCGTAAAAGACGAGCTTAACACCTTGCTTAAGGAGA |
| | | | ATGGCGTAGACTTATCTGTTGTTGTTGAAAAACAAGAAGGC |
| | | | ATGTATAAGAGTGCTCCAAAGCGTCTTACCGCAACAACTGA |
| | | | AAAGTTGGAGATCGGATGGAAAGCTTGGGGAAAATCAATTT |
| | | | TATTTGCACCAGAATTGGCTAACAACACCTTTGTCGTAGACG |
| | | | GTCCTGAGACTAAGGAATGCCCTACCCAGAACAGAGCCTGG |
| | | | AATTCATTAGAAGTCGAGGACTTCGGCTTTGGCCTTACCTCT |
| | | | ACCCGAATGTTCTTAAAAGTTAGAGAGAGTAACACCACAGA |
| | | | ATGCGACAGCAAGATCATTGGCACAGCCGTCAAGAACAATT |
| | | | TAGCTATTCACAGCGATTTGAGCTATTGGATCGAATCTCGCC |
| | | | TTAACGACACATGGAAATTAGAACGCGCAGTATTAGGAGAA |
| | | | GTAAAGTCTTGCACATGGCCTGAAACACACACCTTATGGGG |
| | | | AGATGGCATTCTTGAGAGTGACCTTATTATTCCTGTAACCTT |
| | | | AGCAGGACCACGATCTAATCATAATCGCCGCCCAGGATACA |
| | | | AGACTCAGAACCAGGGTCCTTGGGATGAAGGACGTGTTGAA |
| | | | ATCGACTTTGATTATTGTCCTGGCACCACAGTTACACTTAGT |
| | | | GAGTCTTGTGGTCATCGAGGTCCTGCTACCCGCACTACTACA |
| | | | GAAAGTGGCAAATTGATTACAGACTGGTGCTGTCGATCATG |
| | | | TACATTGCCTCCATTGCGTTACCAGACCGATAGCGGCTGTTG |
| | | | GTATGGTATGGAGATCCGTCCACAACGTCACGATGAGAAAA |
| | | | CTCTTGTACAATCTCAAGTCAATGCTTTCTACCCATCATACC |
| | | | ACTCTACCCCACAACGCCCTTAA |
| 6 | non-structural | codon | GACACCGGTTGCGCCATCGACATTAGTCGACAAGAATTACG |
| | protein 1 (ns1) | optimized | TTGCGGTTCAGGAGTATTCATCCATAACGACGTAGAAGCTTG |
| | | | GATGGACAGATACAAGTACTATCCAGAGACCCCACAGGGAC |
| | | | TTGCAAAGATCATCCAAAAGGCACATAAGGAAGGAGTCTGC |
| | | | GGTCTTAGATCTGTCTCTCGATTGGAGCATCAGATGTGGGAG |
| | | | GCCGTAAAAGACGAGCTTAACACCTTGCTTAAGGAGAATGG |
| | | | CGTAGACTTATCTGTTGTTGTTGAAAAACAAGAAGGCATGT |
| | | | ATAAGAGTGCTCCAAAGCGTCTTACCGCAACAACTGAAAAG |
| | | | TTGGAGATCGGATGGAAAGCTTGGGGAAAATCAATTTTATT |
| | | | TGCACCAGAATTGGCTAACAACACCTTTGTCGTAGACGGTCC |
| | | | TGAGACTAAGGAATGCCCTACCCAGAACAGAGCCTGGAATT |
| | | | CATTAGAAGTCGAGGACTTCGGCTTTGGCCTTACCTCTACCC |
| | | | GAATGTTCTTAAAAGTTAGAGAGAGTAACACCACAGAATGC |
| | | | GACAGCAAGATCATTGGCACAGCCGTCAAGAACAATTTAGC |
| | | | TATTCACAGCGATTTGAGCTATTGGATCGAATCTCGCCTTAA |
| | | | CGACACATGGAAATTAGAACGCGCAGTATTAGGAGAAGTAA |
| | | | AGTCTTGCACATGGCCTGAAACACACACCTTATGGGGAGAT |
| | | | GGCATTCTTGAGAGTGACCTTATTATTCCTGTAACCTTAGCA |
| | | | GGACCACGATCTAATCATAATCGCCGCCCAGGATACAAGAC |
| | | | TCAGAACCAGGGTCCTTGGGATGAAGGACGTGTTGAAATCG |
| | | | ACTTTGATTATTGTCCTGGCACCACAGTTACACTTAGTGAGT |
| | | | CTTGTGGTCATCGAGGTCCTGCTACCCGCACTACTACAGAAA |
| | | | GTGGCAAATTGATTACAGACTGGTGCTGTCGATCATGTACAT |
| | | | TGCCTCCATTGCGTTACCAGACCGATAGCGGCTGTTGGTATG |
| | | | GTATGGAGATCCGTCCACAACGTCACGATGAGAAAACTCTT |
| | | | GTACAATCTCAAGTCAATGCT |

TABLE 2
West Nile Viral amino acid sequences (antigens)

| SEQ ID | | |
|---|---|---|
| NO | Name | Sequence |
| 7 | Premembrane | VTLSNFQGKVMMTVNATDVTDVITIPTAAGKNLCIVRAMDVGYMCDDTIT |
| | (prM) | YECPVLSAGNDPEDIDCWCTKSAVYVRYGRCTKTRHSRRSRR |
| 8 | Envelope (E) | FNCLGMSNRDFLEGVSGATWVDLVLEGDSCVTIMSKDKPTIDVKMMNME |
| | | AANLAEVRSYCYLATVSDLSTKAACPTMGEAHNDKRADPAFVCRQGVVD |
| | | RGWGNGCGLFGKGSIDTCAKFACSTKAIGRTILKENIKYEVAIFVHGPTTVE |
| | | SHGNYSTQVGATQAGRESITPAAPSYTLKLGEYGEVTVDCEPRSGIDTNAY |
| | | YVMTVGTKTFLVHREWFMDLNLPWSSAGSTVWRNRETLMEFEEPHATKQ |
| | | SVIALGSQEGALHQALAGAIPVEFSSNTVKLTSGHLKCRVKMEKLQLKGTT |
| | | YGVCSKAFKFLGTPADTGHGTVVLELQYTGTDGPCKVPISSVASLNDLTPV |
| | | GRLVTVNPFVSVATANAKVLIELEPPFGDSYIVVGRGEQQINHHWHKSGSS |
| | | IGKAFTTTLKGAQRLAALGDTAWDFGSVGGVFTSVGKAVHQVFGGAFRSL |
| | | FGGMSWITQGLLGALLLWMGINARDRSIALTFLAVGGVLLFLSVNVHA |
| 9 | Envelope (E) | FNCLGMSNRDFLEGVSGATWVDLVLEGDSCVTIMSKDKPTIDVKMMNME |
| | residues 1-401 | AANLAEVRSYCYLATVSDLSTKAACPTMGEAHNDKRADPAFVCRQGVVD |
| | | RGWGNGCGLFGKGSIDTCAKFACSTKAIGRTILKENIKYEVAIFVHGPTTVE |
| | | SHGNYSTQVGATQAGRESITPAAPSYTLKLGEYGEVTVDCEPRSGIDTNAY |
| | | YVMTVGTKTFLVHREWFMDLNLPWSSAGSTVWRNRETLMEFEEPHATKQ |
| | | SVIALGSQEGALHQALAGAIPVEFSSNTVKLTSGHLKCRVKMEKLQLKGTT |
| | | YGVCSKAFKFLGTPADTGHGTVVLELQYTGTDGPCKVPISSVASLNDLTPV |
| | | GRLVTVNPFVSVATANAKVLIELEPPFGDSYIVVGRGEQQINHHWHKSG |
| 10 | non-structural | DTGCAIDISRQELRCGSGVFIHNDVEAWMDRYKYYPETPQGLAKIIQKAHK |
| | protein 1 (ns1) | EGVCGLRSVSRLEHQMWEAVKDELNTLLKENGVDLSVVVEKQEGMYKS |
| | | APKRLTATTEKLEIGWKAWGKSILFAPELANNTFVVDGPETKECPTQNRA |
| | | WNSLEVEDFGFGLTSTRMFLKVRESNTTECDSKIIGTAVKNNLAIHSDLSY |
| | | WIESRLNDTWKLERAVLGEVKSCTWPETHTLWGDGILESDLIIPVTLAGPR |
| | | SNHNRRPGYKTQNQGPWDEGRVEIDFDYCPGTTVTLSESCGHRGPATRTT |
| | | TESGKLITDWCCRSCTLPPLRYQTDSGCWYGMEIRPQRHDEKTLVQSQVN |
| | | A |
| 11 | non-structural | DRYKYYPETPQGLAKIIQKAHKEGVCGLRSVSRLEHQMWEAVKDELNTLL |
| | protein 1 (ns1) | KENGVDLSVVVEKQEGMYKSAPKRLTATTEKLEIGWKAWGKSILFAPELA |
| | residues 30- | NNTFVVDGPETKECPTQNRAWNSLEVEDFGFGLTSTRMFLK |
| | 170 | |
| 12 | non-structural | SKIIGTAVKNNLAIHSDLSYWIESRLNDTWKLERAVLGEVKSCTWPETHTL |
| | protein 1 | WGDGILESDLIIPVTLAGPRSNHNRRPGYKTQNQGPWDEGRVEIDFDYCPG |
| | (ns1), residues | TTVTLSESCGHRGPATRTTTE |
| | 181-303 | |

TABLE 3
Nucleic acid sequences for the adjuvants

| SEQ ID NO | Name | Sequence |
|---|---|---|
| 13 | dendritic cell | TTCTACCCATCATACCAC |
| | peptide (DCpep) | TCTACCCCACAACGCCCT |

TABLE 4
Amino acid sequences for the adjuvants

| SEQ ID NO | Name | Sequence |
|---|---|---|
| 14 | dendritic cell | FYPSYHSTPQRP |
| | peptide | |
| | (DCpep) | |

TABLE 5
Plasmids

| SEQ ID | | |
|---|---|---|
| NO | Name | Sequence |
| 15 | pTRK1038 | ACTCGGCACTTAATGGGGGGTCGTAGTACGGAAGCAAAATTCGCTTCCTT |
| | | TCCCCCCATTTTTTTCCAAATTCCAAATTTTTTTCAAAAATTTTCCAGCGCT |
| | | ACCGCTCGGCAAAATTGCAAGCAATTTTTAAAATCAAACCCATGAGGGAA |
| | | TTTCATTCCCTCATACTCCCTTGAGCCTCCTCCAACCGAAATAGAAGGGCG |
| | | CTGCGCTTATTATTTCATTCAGTCATCGGCTTTCATAATCTAACAGACAAC |
| | | ATCTTCGCTGCAAAGCCACGCTACGCTCAAGGGCTTTTACGCTACGATAA |
| | | CGCCTGTTTTAACGATTATGCCGATAACTAAACGAAATAAACGCTAAAAC |
| | | GTCTCAGAAACGATTTTGAGACGTTTTAATAAAAAATCGCTAGTCCGAGG |
| | | CCTCGACCCGATTCACAAAAAATAGGCACACGAAAAACAAGTTAAGGGA |
| | | TGCAGTTTATGCATCCCTTAACTTACTTATTAAATAATTTATAGCTATTGA |
| | | AAAGAGATAAGAATTGTTCAAAGCTAATATTGTTTAAATCGTCAATTCCT |
| | | GCATGTTTTAAGGAATTGTTAAATTGATTTTTTGTAAATATTTTCTTGTATT |
| | | CTTTGTTAACCCATTTCATAACGAAATAATTATACTTTTGTTTATCTTTGTG |
| | | TGATATTCTTGATTTTTTTCTACTTAATCTGATAAGTGAGCTATTCACTTTA |
| | | GGTTTAGGATGAAAATATTCTCTTGGAACCATACTTAATATAGAAATATC |
| | | AACTTCTGCCATTAAAAGTAATGCCAATGAGCGTTTTGTATTTAATAATCT |
| | | TTTAGCAAACCCGTATTCCACGATTAAATAAATCTCATTAGCTATACTATC |
| | | AAAAACAATTTTGCGTATTATATCCGTACTTATGTTATAAGGTATATTACC |
| | | ATATATTTTATAGGATTGGTTTTTAGGAAATTTAAACTGCAATATATCCTT |
| | | GTTTAAAACTTGGAAATTATCGTGATCAACAAGTTTATTTTCTGTAGTTTT |
| | | GCATAATTTATGGTCTATTTCAATGGCAGTTACGAAATTACACCTCTTTAC |
| | | TAATTCAAGGGTAAAATGGCCTTTTCCTGAGCCGATTTCAAAGATATTATC |
| | | ATGTTCATTTAATCTTATATTTGTCATTATTTTATCTATATTATGTTTTGAA |
| | | GTAATAAAGTTTTGACTGTGTTTTATATTTTTCTCGTTCATTATAACCCTCT |
| | | TTAATTTGGTTATATGAATTTTGCTTATTAACGATTCATTATAACCTTACTT |
| | | AGTACCGAAGAGTCTGTCACCGGCATCTCCCAAACCAGGGAAAATGTAA |
| | | CCATTGTCCAGCAATTTATCATCTTCTGATGCAGCATAAATATCAACATCA |
| | | GGATTTTCTTCTTGGACTGCCTTAATACCTTCTGGAGCTGCTACTAAAACT |
| | | GCCAATTTAATATCCTTAACACCACGCTTCTTTAATGCAGCGATAGCGTCA |
| | | TTAGCAGAACCACCAGTAGCAAGCATTGGATCAACAACTAAACATTCACG |
| | | TTCAGCAATATCCTTTGGAGCTTTAAAGAAATATTCATGCGGCTTCAAAGT |
| | | TTCTTCATCACGATACATACCAATAACAGCAATCTTAGCTGATGGTACCAT |
| | | CTCAAGAACACCGTTAAGCATTCCCATACCTGCACGCAAAATCGGCACAA |
| | | TTGTCAATTTCTTGCCGGCGATTTCTTTTTGGACTGTCTTACCCATTGGTGT |
| | | TTCAATTTCAACATCTTCAAGTGGTAAATCTCTAGTAATTTCATAGGTCAT |
| | | TAATCCACCGATTTCACCAACAATTCTACGAAATTCGTTTGAACCTGTGTC |
| | | CTTGCGACGAATAATTGTTAATTTGTGCTGAATCAAAGGGTGATCCAAAA |
| | | CTACAAACTTTCCCATATATGCCTCCTATTAACAAATAACTTAATTCATTA |
| | | AATATTTTAATCAAAAACGCAAAGGGTTTAAAGCCCTTTGCGTTTTATTTT |
| | | TTATTTATTCAAATGATGGCCGCCGGTGAAAACCTCTGACACATGCAGCT |
| | | CCCGGAGACGGTCACAGCTTGTCTGTAAGCGGATGCCGGGAGCAGACAA |
| | | GCCCGTCAGGGCGCGTCAGCGGGTGTTGGCGGGTGTCGGGGCTGGCTTAA |
| | | CTATGCGGCATCAGAGCAGATTGTACTGAGAGTGCACCATATGCGGTGTG |
| | | AAATACCGCACAGATGCGTAAGGAGAAAATACCGCATCAGGCGCCATTC |
| | | GCCATTCAGGCTGCGCAACTGTTGGGAAGGGCGATCGATAGCGATTGTAT |
| | | TAGTAACTAATTTATTAGCTGGATGTAGTCAAATCCATTTTGGCAAAGAT |
| | | GCAATCACAATTGGTGATGAGCAAAAAAATCTTTTAAAGAAGAATAAAA |
| | | AGAAAAAAGTAGTACATCCTAAAATTGAAAAGAAAAAAACAAAAGTAAA |
| | | AAGTGTTTGGAACAAAACAAAGTATCAGGAATTACAAAAGATTGTAAAT |
| | | AATTGGGGTAGGGTAAAAAAACAACATTATCATTTTTATGATGGCGTCCA |
| | | TTCTTTAAAAATAAAAGCAGGAGTTACATATCCAAAAGCTTTTGATCAAA |
| | | ATGGTTTCGTTTTAAATAAAGAAAAAATCAAAATAGGGTGGAGTCCAGAA |
| | | GGTAAAAATACGTATCAATACAATGTTGTAGCAATTGCCAATGATAATTT |
| | | TGAAACATGGCACAATACATATTTGTTTTGTTTAAGAAAGAATAAACCAA |
| | | TTATTTTATTAGATCAGTCAAAACGAAATAAGCTTGTATTAGTTAAGAAA |
| | | GTGAATAATCCTACTCTTAATAAAGCCTTTAAACAAATTTTAGAAGATAA |
| | | ACAAAAAAGCATGTAGAAAATTAATCTACATGCTTTTTGTTGAAATTGTA |
| | | CTCGGTGCGGCCGCATTAATCGTGGTCAACATCATCTGGGAAGAATGCAA |
| | | GACGTTCGCCCTTGCCAAGTTCTTCTTCGATTTCAAGTAAGCGGTTGTACT |
| | | TTTCAACACGTTCTGAACGAGCTGGAGCACCGGTCTTAAGTTGACCACCG |
| | | TTTATAGCAACTGCCAAGTCAGCGATGAATGTGTCACCAGTTTCACCTGA |
| | | ACGGTGTGAAATCATAGTGTTGTAACCATTCTTACGTGACATACGGATAG |
| | | TTTCAAGAGTTTCAGTAACAGTACCAATTTGGTTCAACTTGATTAATGAAG |
| | | CGTTACCTGCACCTTCCTTGATAGCCTTCTTCAAAAGTGCTGGGTTAGTAC |
| | | AAATGAAGTCGTCAAGAACGATTTGGATACGGTCCTTGTGAGTTGCAGTA |
| | | AACTTAACCATACCTTCAACATCGTTTTCGTCGTATGGGTCTTCCATTGAC |
| | | ATTAATTCTGGGAATTCATCCAAAAGCTTGTCGTAGTATGCAGCTAATTCA |
| | | TCATCAGTAAGAACCTTACCTTCAAGGTGGTACTTACCATCTTCCTTGTTG |
| | | TAGAAGTATGAAGCAGCACAGTCACATGCGATACCGATATCTTCACCTGG |
| | | CTTGTAACCAGCCTTGATGATTGATTCGTGTAAAGTCGACCTGCAGGCAT |
| | | GCAAGCTTGGCGTAATCATGGTCATAGCTGTTTCCTGTGTGAAATTGTTAT |
| | | CCGCTCACAATTCCACACAACATACGAGCCGGAAGCATAAAGTGTAAAG |
| | | CCTGGGGTGCCTAATGAGTGAGCTAACTCACATTAATTGCGTTGCGCTCA |
| | | CTGCCCGCTTTCCAGTCGGGAAACCTGTCGTGCCAGCTGCATTAATGAAT |
| | | CGGCCAACGCGCGGGGAGAGGCGGTTTGCGTATTGGGCGCTCTTCCGCTT |
| | | CCTCGCTCACTGACTCGCTGCGCTCGGTCGTTCGGCTGCGGCGAGCGGTA |
| | | TCAGCTCACTCAAAGGCGGTAATACGGTTATCCACAGAATCAGGGGATAA |
| | | CGCAGCTAGTTCTAGAACTAGCGATTCTGAAATCACCATTTAAAAAACTC |
| | | CAATCAAATAATTTTATAAAGTTAGTGTATCACTTTGTAATCATAAAAAC |
| | | AACAATAAAGCTACTTAAATATAGATTTATAAAAAACGTTGGCGAAAACG |
| | | TTGGCGATTCGTTGGCGATTGAAAAACCCCTTAAACCCTTGAGCCAGTTG |
| | | GGATAGAGCGTTTTTGGCACAAAAATTGGC |
| 16 | pTRK669 | GACTGCTAGCAGCGAAGATGTTGTCTGTTAGATTATGAAAGCCGATGACT |
| | | GAATGAAATAATAAGCGCAGCGCCCTTCTATTTCGGTTGGAGGAGGCTCA |
| | | AGGGAGTATGAGGGAATGAAATTCCCTCATGGGTTTGATTTTAAAAATTG |
| | | CTTGCAATTTTGCCGAGCGGTAGCGCTGGAAAATTTTTGAAAAAAATTTG |
| | | GAATTTGGAAAAAAATGGGGGGAAAGGAAGCGAATTTTGCTTCCGTACT |
| | | ACGACCCCCCATTAAGTGCCGAGTGCCAATTTTTGTGCCAAAAACGCTCT |
| | | ATCCCAACTGGCTCAAGGGTTTAAGGGGTTTTTCAATCGCCAACGAATCG |
| | | CCAACGTTTTCGCCAACGTTTTTTATAAATCTATATTTAAGTAGCTTTATT |
| | | GTTGTTTTTATGATTACAAAGTGATACACTAACTTTATAAAATTATTTGAT |
| | | TGGAGTTTTTTAAATGGTGATTTCAGAATCGAAAAAAAGAGTTATGATTT |
| | | CTCTGACAAAAGAGCAAGATAAAAAATTAACAGATATGGCGAAACAAAA |
| | | AGGTTTTTCAAAATCTGCGGTTGCGGCGTTAGCTATAGAAGAATATGCAA |
| | | GAAAGGAATCAGAACAAAAAAAATAAGCGAAAGCTCGCGTTTTTAGAAG |
| | | GATACGAGTTTTCGCTACTTGTTTTTGATAAGGTAATTATATCATGGCTAT |
| | | TAAAAATACTAAAGCTAGAAATTTTGGATTTTTATTATATCCTGACTCAAT |
| | | TCCTAATGATTGGAAAGAAAAATTAGAGAGTTTGGGCGTATCTATGGCTG |
| | | TCAGTCCTTTACACGATATGGACGAAAAAAAAGATAAAGATACATGGAA |
| | | TAGTAGTGATGTTATACGAAATGGAAAGCACTATAAAAAACCACACTATC |
| | | ACGTTATATATATTGCACGAAATCCTGTAACAATAGAAAGCGTTAGGAAC |
| | | AAGATTAAGCGAAAATTGGGGAATAGTTCAGTTGCTCATGTTGAGATACT |
| | | TGATTATATCAAAGGTTCATATGAATATTTGACTCATGAATCAAAGGACG |
| | | CTATTGCTAAGAATAAACATATATACGACAAAAAAGATATTTTGAACATT |
| | | AATGATTTTGATATTGACCGCTATATAACACTTGATGAAAGCCAAAAAAG |
| | | AGAATTGAAGAATTTACTTTTAGATATAGTGGATGACTATAATTTGGTAA |
| | | ATACAAAAGATTTAATGGCTTTTATTCGCCTTAGGGGAGCGGAGTTTGGA |
| | | ATTTTAAATACGAATGATGTAAAAGATATTGTTTCAACAAACTCTAGCGC |
| | | CTTTAGATTATGGTTTGAGGGCAATTATCAGTGTGGATATAGAGCAAGTT |
| | | ATGCAAAGGTTCTTGATGCTGAAACGGGGGAAATAAAATGACAAACAAA |
| | | GAAAAAGAGTTATTTGCTGAAAATGAGGAATTAAAAAAAGAAATTAAGG |
| | | ACTTAAAAGAGCGTATTGAAAGATACAGAGAAATGGAAGTTGAATTAAG |
| | | TACAACAATAGATTTATTGAGAGGAGGGATTATTGAATAAATAAAAGCCC |
| | | CCTGACGAAAGTCGAAGGGGGTTTTTATTTTGGTTTGATGTTGCGATTAAT |
| | | AGCAATACAATTGCAATAAACAAAATGATCTTCCTTCAGGTTATGACCAT |
| | | CTGTGCCAGTTCGTAATGTCTGGTCAACTTTCCGACTCTGAGAAACTTCTG |
| | | GAATCGCTAGAGAATTTCTGGAATGGGATTCAGGAGTGGACAGAACGAC |
| | | ACGGATATATAGTGGATGTGTCAAAACGCATACCATTTTGAACGATGACC |
| | | TCTAATAATTGTTAATCATGTTGGTTACGTATTTATTAACTTCTCCTAGTAT |
| | | TAGTAATTATCATGGCTGTCATGGCGCATTAACGGAATAAAGGGTGTGCT |
| | | TAAATCGGGCCATTTTGCGTAATAAGAAAAAGGATTAATTATGAGCGAAT |
| | | TGAATTAATAATAAGGTAATAGATTTACATTAGAAAATGAAAGGGGATTT |
| | | TATGCGTGAGAATGTTACAGTCTATCCCGGCAATAGTTACCCTTATTATCA |
| | | AGATAAGAAAGAAAAGGATTTTTCGCTACGCTCAAATCCTTTAAAAAAAC |
| | | ACAAAAGACCACATTTTTTAATGTGGTCTTTATTCTTCAACTAAAGCACCC |
| | | ATTAGTTCAACAAACGAAAATTGGATAAAGTGGGATATTTTTAAAATATA |
| | | TATTTATGTTACAGTAATATTGACTTTTAAAAAAGGATTGATTCTAATGAA |
| | | GAAAGCAGACAAGTAAGCCTCCTAAATTCACTTTAGATAAAAATTTAGGA |
| | | GGCATATCAAATGAACTTTAATAAAATTGATTTAGACAATTGGAAGAGAA |
| | | AAGAGATATTTAATCATTATTTGAACCAACAAACGACTTTTAGTATAACC |
| | | ACAGAAATTGATATTAGTGTTTTATACCGAAACATAAAACAAGAAGGATA |
| | | TAAATTTTACCCTGCATTTATTTTCTTAGTGACAAGGGTGATAAACTCAAA |
| | | TACAGCTTTTAGAACTGGTTACAATAGCGACGGAGAGTTAGGTTATTGGG |
| | | ATAAGTTAGAGCCACTTTATACAATTTTTGATGGTGTATCTAAAACATTCT |
| | | CTGGTATTTGGACTCCTGTAAAGAATGACTTCAAAGAGTTTTATGATTTAT |
| | | ACCTTTCTGATGTAGAGAAATATAATGGTTCGGGGAAATTGTTTCCCAAA |
| | | ACACCTATACCTGAAAATGCTTTTTCTCTTTCTATTATTCCATGGACTTCAT |
| | | TTACTGGGTTTAACTTAAATATCAATAATAATAGTAATTACCTTCTACCCA |
| | | TTATTACAGCAGGAAAATTCATTAATAAAGGTAATTCAATATATTTACCG |
| | | CTATCTTTACAGGTACATCATTCTGTTTGTGATGGTTATCATGCAGGATTG |
| | | TTTATGAACTCTATTCAGGAATTGTCAGATAGGCCTAATGACTGGCTTTTA |
| | | TAATATGAGATAATGCCGACTGTACTTTTTACAGTCGGTTTTCTAATGTCA |
| | | CTTCTAGATCTG |
| 17 | pTRK1053 | AGCTTCATCTGAGGATAAAGTTGTTTGATAAATGCTCAACTTTAAGTAATT |
| | | TTAAGGAGCTAACTAACTGTGGGGGATGAAATAAAGCCAATAGAAAAAG |
| | | CGAACCTAATAAGATTAATCTTTAGGAAAATCGAATAAAAATATTACTTT |
| | | TTTGATATGTTTTGTCATAGTTTCGTAAAATTTAGTAAAGATTACGAGCGA |
| | | TAAATAGAGAACTTAATCTTGTCTTTTTCTTGCTATAGCTAGGTTTAGCAC |
| | | ATTTTACAATTTTAAAGTGCTTGTAATGCTTGTGGGGGTAAGCGGTAGGT |
| | | GAAATATTACAAATAGTATTTTTCGGTCATTTTAACTTGCTATTTCTTGAA |
| | | GAGGTTAGTACAATATGAATCGTGGTAAGTAATAGGACGTGCTTCAGGCG |
| | | TGTTGCCTGTACGCATGCTGATTCTTCAGCAAGACTACTACCTCATGAGAG |
| | | TTATAGACTCATGGATCTTGCTTTGAAGGGTTTTGTACATTATAGGCTCCT |
| | | ATCACATGCTGAACCTATGGCCTATTACATTTTTTTATATTTCAAGGAGGA |
| | | AAAGACCACATGAAGAAAAATTTAAGAATCGTTAGCGCTGCTGCTGCTGC |
| | | TTTACTTGCTGTTGCTCCAGTTGCTGCTTCTGCTGTATCTACTGTTAGCGCT |
| | | GCTACTACTATTAACGCAAGTTCATCAGCAATCAATACCAACACTAATGC |
| | | TAAGTACGATGTTGATGTAACTCCTAGTGTTTCTGCAGTTGCTGCAAATAC |
| | | TGCTAACAACACTCCAGCTATTGCCGGTAACCTTACTGGTACTATTTCAGC |
| | | AAGTTACAATGGTAAGACTTATACTGCTAACTTAAAGGCAGATACTGAAA |
| | | ATGCCACTATTACTGCTGCTGGTAGCACTACTGCCGTTAAACCTGCTGAAT |
| | | TAGCTGCAGGTGTGGCTTACACTGTAACTGTTAACGATGTTTCATTTAACT |
| | | TCGGTTCAGAAAATGCAGGTAAGACTGTTACCCTTGGTTCAGCTAACTCA |
| | | AATGTAAAATTCACCGGTACAAACAGTGATAATCAAACTGAAACTAATGT |
| | | TTCTACTTTGAAAGTTAAGTTAGACCAAAACGGTGTTGCTTCACTTACTAA |
| | | TGTTTCAATTGCAAACGTATACGCAATTAACACTACTGATAACAGTAACG |
| | | TAAACTTCTACGACGTAACTAGTGGTGCTACTGTAACTAACGGTGCCGTTT |
| | | CAGTTAATGCTGATAACCAAGGTCAAGTTAATGTTGCAAACGTAGTTGCA |
| | | GCAATTAATTCAAAATACTTTGCAGCACAATACGCAGATAAGAAGTTAAA |
| | | TACTCGTACTGCTAATACTGAAGATGCTATTAAGGCAGCCTTAAAGGACC |
| | | AAAAGATTGATGTAAACTCAGTAGGTTACTTCAAAGCACCTCATACTTTC |
| | | ACTGTTAACGTTAAAGCAACTTCAAATACTAATGGTAAGTCAGCTACTTT |
| | | GCCAGTAGTTGTTACTGTTCCTAATGTTGCTGAGCCAACTGTAGCCAGCGT |
| | | AAGCAAGAGAATTATGCACAACGCATACTACTACGACAAGGACGCTAAG |
| | | CGTGTTGGTACTGACAGCGTTAAGCGTTACAACTCAGTAAGCGTATTGCC |
| | | AAACACTACTACTATCAACGGTAAGACTTACTACCAAGTAGTTGAAAACG |
| | | GTAAGGCTGTTGACAAGTACATCAACGCTGCAAACATCGATGGTACTAAG |
| | | CGTACTTTGAAGCACAACGCTTACGTTTACGCATCATCAAAGAAGCGTGC |
| | | TAACAAGGTTGTATTGAAGAAGGGTGAAGTTGTAACTACTTACGGTGCTT |
| | | CATACACATTCAAGAACGGCCAAAAGTACTACAAGATCGGTGACAACAC |
| | | TGACAAGACTTACGTTAAGGTTGCAAACTTTAGATAATAAGTCGTAGCAC |
| | | TAACGCTAACAAAATGAAAAAGGCAGAGCGAAAGCTCTGTCTTTTTTTGT |
| | | GTTTTAAATTTTTATTTCACTTCTAAACATACAGTTCTAATAGGATCTTCTG |
| | | ATACTTCGATTCGGATCCCCGGGTACCGAGCTCGAATTCACTGGCCGTCG |
| | | TTTTACAACGTCGTGACTGGGAAAACCCTGGCGTTACCCAACTTAATCGC |
| | | CTTGCAGCACATCCCCCTTTCGCCAGCTGGCGTAATAGCGAAGAGGCCCG |
| | | CACCGATCGCCCTTCCCAACAGTTGCGCAGCCTGAATGGCGAATGGCGCC |
| | | TGATGCGGTATTTTCTCCTTACGCATCTGTGCGGTATTTCACACCGCATAT |
| | | GGTGCACTCTCAGTACAATCTGCTCTGATGCCGCATAGTTAAGCCAGCCC |
| | | CGACACCCGCCAACACCCGCTGACGCGCCCTGACGGGCTTGTCTGCTCCC |
| | | GGCATCCGCTTACAGACAAGCTGTGACCGTCTCCGGGAGCTGCATGTGTC |
| | | AGAGGTTTTCACCGGCGGCCATCATTTGAATAAATAAAAAATAAAACGCA |
| | | AAGGGCTTTAAACCCTTTGCGTTTTTGATTAAAATATTTAATGAATTAAGT |
| | | TATTTGTTAATAGGAGGCATATATGGGAAAGTTTGTAGTTTTGGATCACCC |
| | | TTTGATTCAGCACAAATTAACAATTATTCGTCGCAAGGACACAGGTTCAA |
| | | ACGAATTTCGTAGAATTGTTGGTGAAATCGGTGGATTAATGACCTATGAA |
| | | ATTACTAGAGATTTACCACTTGAAGATGTTGAAATTGAAACACCAATGGG |
| | | TAAGACAGTCCAAAAAGAAATCGCCGGCAAGAAATTGACAATTGTGCCG |
| | | ATTTTGCGTGCAGGTATGGGAATGCTTAACGGTGTTCTTGAGATGGTACC |
| | | ATCAGCTAAGATTGCTGTTATTGGTATGTATCGTGATGAAGAAACTTTGA |
| | | AGCCGCATGAATATTTCTTTAAAGCTCCAAAGGATATTGCTGAACGTGAA |
| | | TGTTTAGTTGTTGATCCAATGCTTGCTACTGGTGGTTCTGCTAATGACGCT |
| | | ATCGCTGCATTAAAGAAGCGTGGTGTTAAGGATATTAAATTGGCAGTTTT |
| | | AGTAGCAGCTCCAGAAGGTATTAAGGCAGTCCAAGAAGAAAATCCTGAT |
| | | GTTGATATTTATGCTGCATCAGAAGATGATAAATTGCTGGACAATGGTTA |
| | | CATTTTCCCTGGTTTGGGAGATGCCGGTGACAGACTCTTCGGTACTAAGTA |
| | | AGGTTATAATGAATCGTTAATAAGCAAAATTCATATAACCAAATTAAAGA |
| | | GGGTTATAATGAACGAGAAAAATATAAAACACAGTCAAAACTTTATTACT |
| | | TCAAAACATAATATAGATAAAATAATGACAAATATAAGATTAAATGAAC |
| | | ATGATAATATCTTTGAAATCGGCTCAGGAAAAGGCCATTTTACCCTTGAA |
| | | TTAGTAAAGAGGTGTAATTTCGTAACTGCCATTGAAATAGACCATAAATT |
| | | ATGCAAAACTACAGAAAATAAACTTGTTGATCACGATAATTTCCAAGTTT |
| | | TAAACAAGGATATATTGCAGTTTAAATTTCCTAAAAACCAATCCTATAAA |
| | | ATATATGGTAATATACCTTATAACATAAGTACGGATATAATACGCAAAAT |
| | | TGTTTTTGATAGTATAGCTAATGAGATTTATTTAATCGTGGAATACGGGTT |
| | | TGCTAAAAGATTATTAAATACAAAACGCTCATTGGCATTACTTTTAATGG |
| | | CAGAAGTTGATATTTCTATATTAAGTATGGTTCCAAGAGAATATTTTCATC |
| | | CTAAACCTAAAGTGAATAGCTCACTTATCAGATTAAGTAGAAAAAAATCA |
| | | AGAATATCACACAAAGATAAACAAAAGTATAATTATTTCGTTATGAAATG |
| | | GGTTAACAAAGAATACAAGAAAATATTTACAAAAAATCAATTTAACAATT |
| | | CCTTAAAACATGCAGGAATTGACGATTTAAACAATATTAGCTTTGAACAA |
| | | TTCTTATCTCTTTTCAATAGCTATAAATTATTTAATAAGTAAGTTAAGGGA |
| | | TGCATAAACTGCATCCCTTAACTTGTTTTTCGTGTGCCTATTTTTTGTGAAT |
| | | CGGGTCGAGGCCTCGGACTAGCGATTTTTTATTAAAACGTCTCAAAATCG |
| | | TTTCTGAGACGTTTTAGCGTTTATTTCGTTTAGTTATCGGCATAATCGTTA |
| | | AAACAGGCGTTATCGTAGCGTAAAAGCCCTTGAGCGTAGCGTGGCTTTGC |
| | | AGCGAAGATGTTGTCTGTTAGATTATGAAAGCCGATGACTGAATGAAATA |
| | | ATAAGCGCAGCGCCCTTCTATTTCGGTTGGAGGAGGCTCAAGGGAGTATG |
| | | AGGGAATGAAATTCCCTCATGGGTTTGATTTTAAAAATTGCTTGCAATTTT |
| | | GCCGAGCGGTAGCGCTGGAAAATTTTTGAAAAAAATTTGGAATTTGGAAA |
| | | AAAATGGGGGGAAAGGAAGCGAATTTTGCTTCCGTACTACGACCCCCCAT |
| | | TAAGTGCCGAGTGCCAATTTTTGTGCCAAAAACGCTCTATCCCAACTGGC |
| | | TCAAGGGTTTAAGGGGTTTTTCAATCGCCAACGAATCGCCAACGTTTTCG |
| | | CCAACGTTTTTTATAAATCTATATTTAAGTAGCTTTATTGTTGTTTTTATGA |
| | | TTACAAAGTGATACACTAACTTTATAAAATTATTTGATTGGAGTTTTTTAA |
| | | ATGGTGATTTCAGAATCGCTAGTTCTAGAACTAGCTGCGTTATCCCCTGAT |
| | | TCTGTGGATAACCGTATTACCGCCTTTGAGTGAGCTGATACCGCTCGCCGC |
| | | AGCCGAACGACCGAGCGCAGCGAGTCAGTGAGCGAGGAAGCGGAAGAG |
| | | CGCCCAATACGCAAACCGCCTCTCCCCGCGCGTTGGCCGATTCATTAATG |
| | | CAGCTGGCACGACAGGTTTCCCGACTGGAAAGCGGGCAGTGAGCGCAAC |
| | | GCAATTAATGTGAGTTAGCTCACTCATTAGGCACCCCAGGCTTTACACTTT |
| | | ATGCTTCCGGCTCGTATGTTGTGTGGAATTGTGAGCGGATAACAATTTCAC |
| | | ACAGGAAACAGCTATGACCATGATTACGCCA |

### EXAMPLES

### Example 1

### Methods of Genetic Integration of the Adjuvant into Recombinant Lactobacillus Acidophilus

A strain of Lactobacillus acidophilus (NKC56) was successfully transformed to express dendritic cell (DC) immunostimulating adjuvant peptide (DCpep) adjuvant within the surface layer protein A (slpA) gene expressed on the bacteria's surface as further detailed in the following Example. DCpep is a known adjuvant peptide that binds to a DC ligand conserved across vertebrate orders (human, avian, equine, etc), enhancing DC activation, maturation and stimulation of B- and T-cell adaptive immune responses against the foreign antigens produced in the rLA.

The full length dendritic cell (DC) immunostimulating adjuvant peptide (DCpep) was integrated into the chromosome of Lactobacillus acidophilus (LA) using a developed upp-based counter selective gene replacement system and two previously constructed chromosomal integration plasmids. The constructed chromosomal integration plasmid was pTRK1053, containing the entire 1,335 bp surface layer protein A (slpA) gene.

The DCpep cassette was generated by designing two extension primers (BS34 and BS35) to add overlapping sequences encoding DCpep into SlpA. The resultant amplicons were combined using overlap PCR, then assembled into pTRK1053 using the Instant Sticky-end Ligase Master Mix (New England BioLabs, NEB, Ipswich, MA, USA) to generate DCpep integration plasmid pTRK[dcp]. The reaction mixture was then heat-shock transformed into cloning host E. coli EC101 for amplification. The transformants were plated on BHI agar plates supplemented with 40 μg/mL kanamycin and 150 μg/mL erythromycin (Erm) and incubated at 37° C. Resulting colonies were screened with primers AK63 and AK64 for positive transformants and confirmed by Sanger sequencing. Plasmid pTRK[dcp] was then extracted from positive transformants and electroporated into LA strain NCK1910, which also contains plasmid pTRK669 (provides repA in trans for the replication of pTRK[dcp]).

Selection for single-crossover DCpep recombinants was then performed. Briefly, single-crossover recombinants were selected after growing the NCK1910 transformants at 42° C. for 72 hours in MRS supplemented with 5 μg/mL Erm. Single-crossover plasmid integration was confirmed by screening with primers AK62 and BS37. The single-crossover recombinants were then grown at 37° C. for 72 hours without antibiotic pressure to facilitate the double-crossover recombination event. The double-crossover recombinants were selected with 100 g/mL 5-fluorouracil (Sigma-Aldrich, St. Louis, MO, USA). The chromosomal integration (double-crossover) mutants were confirmed by PCR using primers AK62 and AK65 and Sanger sequencing. The resulting strain expressing the DCpep cassette was designated LA GAD303 (rLA-DCpep).

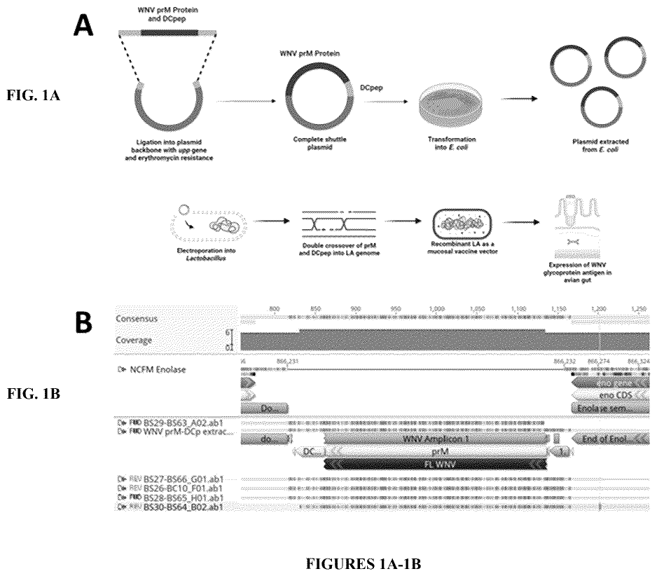

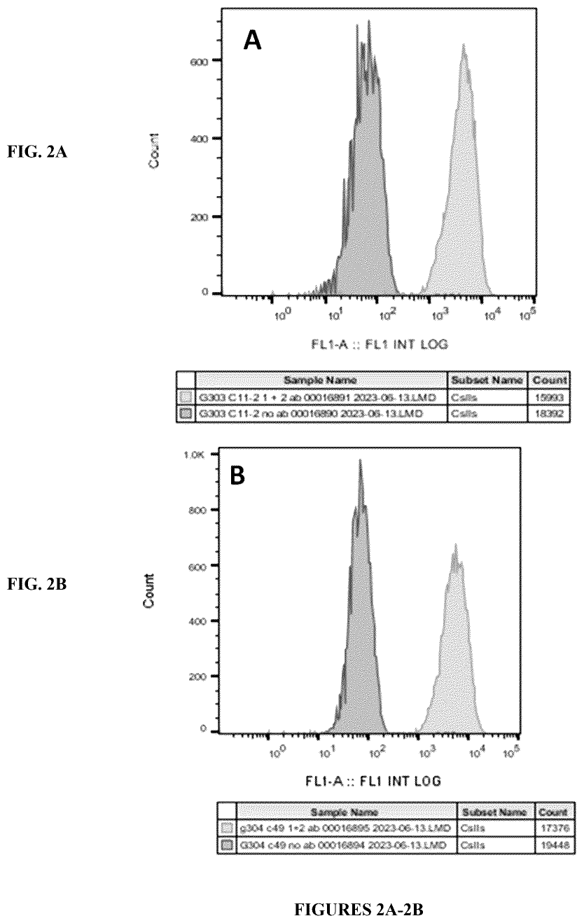

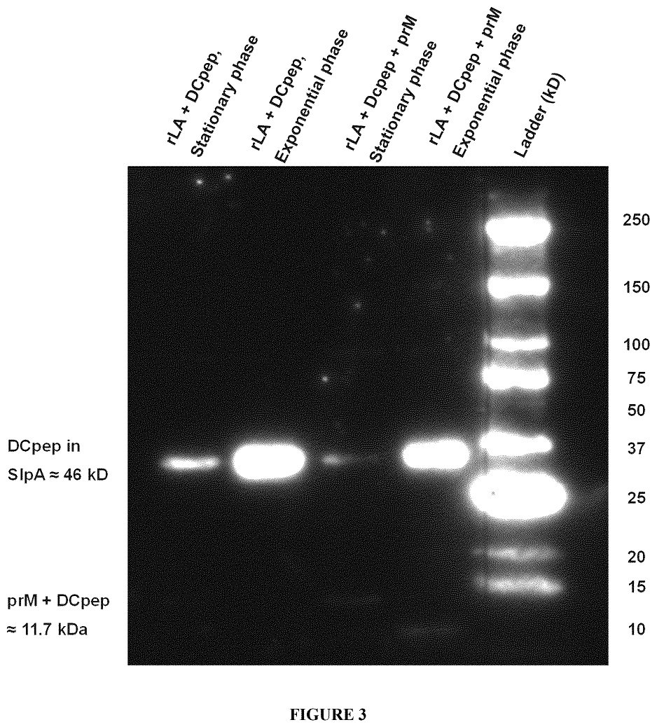

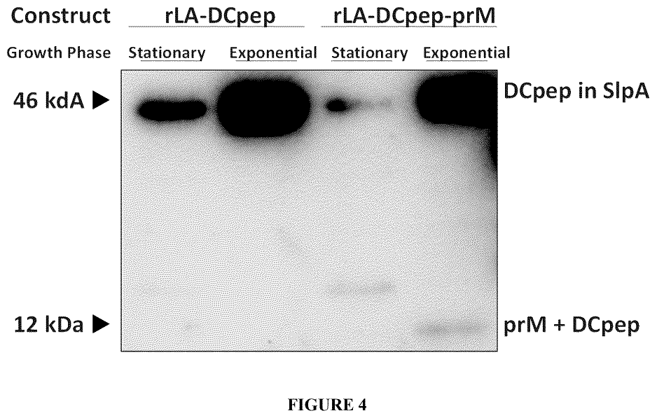

To confirm integration, colonies were screened by PCR of the chromosomal sequence to show replacement of the wild-type band with that of the edited insert. Insertion fidelity was additionally confirmed by Sanger sequencing of the PCR-positive colonies (FIG. 1B). Cell surface adjuvant expression was validated by FACS (FIG. 2A, FIG. 2B), and cytosolic antigen expression validated by Western blot of rLA-WNV cell growths (FIGS. 3-4). LA media and wild-type LA represented negative controls and heat-inactivated WNV harvested from Vero cell culture positive controls throughout. Stability was assessed by comparing expression of inserts between low and high passage number isolates, and between freshly passaged vs. frozen isolates.

### Example 2

### Methods of Genetic Integration of Exemplary Antigens and Adjuvants into Recombinant Lactobacillus acidophilus

Several in vitro and in vivo cloning strategies have been developed and validated to put key WNV genes in rLA. An example workflow is shown in FIG. 1A, which demonstrates standard gene cloning techniques to create plasmids (either in E. coli or in vitro via rolling circle amplification) containing WNV genes or gene fragments. Additionally, constructs where the WNV genes were fused to a dendritic cell (DC) immunostimulating adjuvant peptide (DCpep) were generated. Each type of rLA construct was generated so that the nucleotide sequence of the WNV gene was flanked by nucleotides allowing for genomic insertion behind the highly expressed chromosomal housekeeping gene enolase, allowing for cytosolic accumulation of the protein in the exponential growth phase of rLA.

Two WNV antigens were pursued to develop a probiotic strain for immunization. The premembrane protein (prM), and nonstructural protein 1 (NS1) were integrated into the plasmid pTRK1038 containing 600 bp of upstream and 600 bp of downstream sequences flanking the immediate downstream site of the highly expressed gene lba0889 (enolase).

Each cassette was independently assembled with PCR-amplified pTRK1038 backbone with added overlap sequences using NEBuilder HiFi DNA Assembly mix (New England BioLabs, NEB, Ipswich, MA, USA) to generate two unique plasmids. The first plasmid, a prM integration plasmid, pTRK[prm] and the second plasmid for NS1 integration, pTRK [ns1]. These were then independently electroporated into cloning host E. coli (EC101) for amplification, then into (GAD303, the previously generated rLA strain containing the adjuvant DC peptide, DCpep) for chromosomal integration. The prM-DCpep and NS1-DCpep integration and selection procedures follow similar steps as described in Example 1 for DCpep in slpA, and briefly detailed below.

The prM, ns1, prM-DCpep, ns1-DCpep constructs were generated by designing two extension primers to add overlapping sequences encoding prM and ns1 into the enolase gene. The resultant amplicons were combined using overlap PCR, then assembled into pTRK1053 using the Instant Sticky-end Ligase Master Mix (New England BioLabs, NEB, Ipswich, MA, USA) to generate each of the prM and ns1 integration plasmids pTRK [prM] and pTRK [ns1] plasmids, respectively. The reaction mixture was then heat-shock transformed into cloning host E. coli EC101 for amplification. The transformants were plated on BHI agar plates supplemented with 40 μg/mL kanamycin and 150 μg/mL erythromycin (Erm) and incubated at 37° C. Resulting colonies were screened with primers for positive transformants and confirmed by Sanger sequencing (FIG. 1B). Plasmid pTRK [prM] and pTRK [ns1] were extracted from positive transformants and electroporated into LA strains.

The rLA construct containing prM and DCpep was generated by amplifying prM using extension primers BS38 and BS39 to add DCpep to the 3′ end of prM derived from the WNV infectious clone. The NS1-DCpep cassette was obtained as a clonal gene (Twist Biosciences, San Francisco, CA) with overlap sequences for downstream assembly.

Selection for single-crossover prM and ns1 recombinants were then performed. Briefly, single-crossover recombinants were selected after growing the transformants at 42° C. for 72 hours in MRS supplemented with 5 μg/mL Erm. Single-crossover plasmid integration was confirmed by screening with primers. The single-crossover recombinants were then grown at 37° C. for 72 hours without antibiotic pressure to facilitate the double-crossover recombination event. The double-crossover recombinants were selected with 100 μg/mL 5-fluorouracil (Sigma-Aldrich, St. Louis, MO, USA). The chromosomal integration (double-crossover) mutants were confirmed by PCR using primers and Sanger sequencing. Additionally, the resulting strains expressing both DCpep in slpA and prM-DCpep downstream of enolase was designated GAD304 (rLA-DCpep-prM), and the resulting strain expressing both DCpep in slpA and NS1-DCpep downstream of enolase was designated GAD305 (rLA-DCpep-NS1).

The sequences were validated and the plasmid constructs were electroporated into rLA-DCpep. The constructs for rLA-DCpep-prM (GAD304) and rLA-DCpep-ns1 (GAD305) were completed and validated. Notably, rLA-DCpep-prM expresses DC-pep on the cell surface and DC-pep+prM in the cytoplasm (FIG. 2A, FIG. 2B, FIG. 3 and FIG. 4). Furthermore, a proteomics analysis validated the expression of West Nile virus non-structural protein 1 (NS1) expressed in the rLA bacterium.

Construction of the envelope (E) expressing constructs can be difficult due to bacterial toxicity issues when propagating plasmids in bacteria due to the presence of cryptic bacterial promoters in flaviviral sequences. As alternative techniques, in vitro rolling circle amplification can be used to avoid relying on bacteria to replicate necessary concentrations of plasmid to proceed with transformation into LA. Other options are to order gene blocks for cloning containing several immunostimulatory epitopes of the E domain III or a concatenation of these peptide sequences with flexible intragenic spacers.

### Example 3

### Microencapsulation and Lyophilization of Recombinant Lactobacillus acidophilus

Validation of the generated rLA cultures as described in Example 1 and Example 2 were intended for rapid validation and comparison of the immunity that the generated rLA constructs were expected to stimulate in the different bird species.

Microencapsulation and lyophilization of L. acidophilus results in a shelf-stable product that can be easily transported without the need for complicated storage and cold chain. By overcoating and/or fixing the lyophilized rLA construct to bird feed, lyophilized bacterial constructs may be further protected from environmental factors, creating an easy and effective means for immunizing wild or domesticated bird populations.

Immunization with lyophilized rLA bound to feed would be increasingly beneficial for practical applications in the field. Further to this end, the developed lyophilized rLA constructs generated in Examples 1 and 2 were used for developing lyophilized bird feed formulation combinations using the most the successful rLA constructs. The goal of this Example was to successfully microencapsulate rLA constructs that stimulate antigen specific immunity and bind the rLA (and microencapsulated rLA) to bird feed.

Lyophilization of Wildtype L. acidophilus and Recombinant L. acidophilus

Wildtype Lactobacillus acidophilus (NCK56) was used to develop the lyophilization protocol. Previously published protocols were used for the specific wildtype LA and rLA constructs. The first step was to ensure wild-type LA were able to be successfully lyophilized and maintain high viability. Next, the developed protocol used for the wildtype LA was applied to each of the validated rLA constructs that exhibited robust recombinant vaccine antigen expression. The lyophilized samples were then analyzed using Scanning Electron Microscopy (SEM) and flow cytometry to assess any physical and physiological changes of the bacteria following lyophilization and ensure sustained expression of the respective proteins incorporated into the rLA strain.

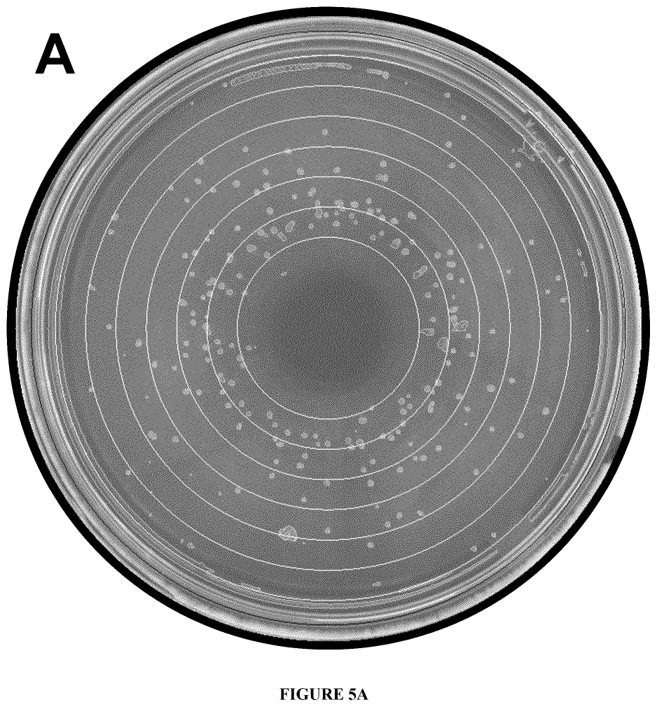

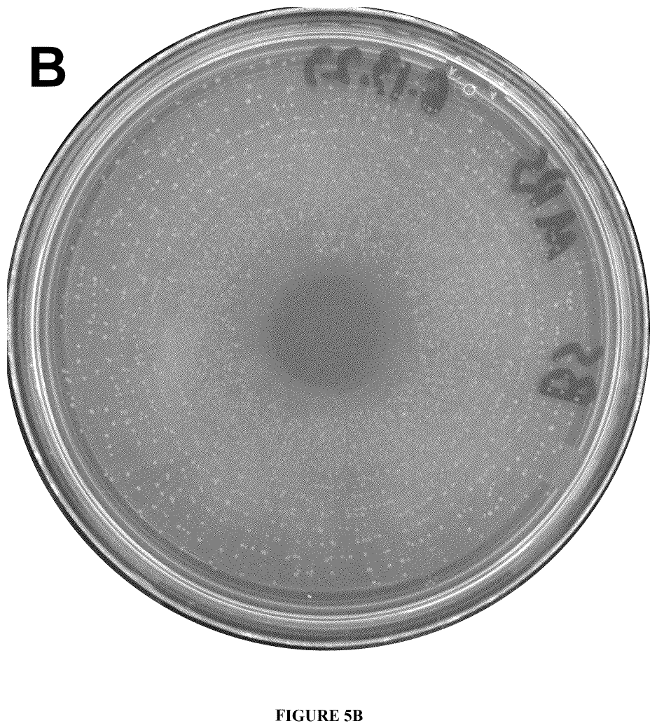

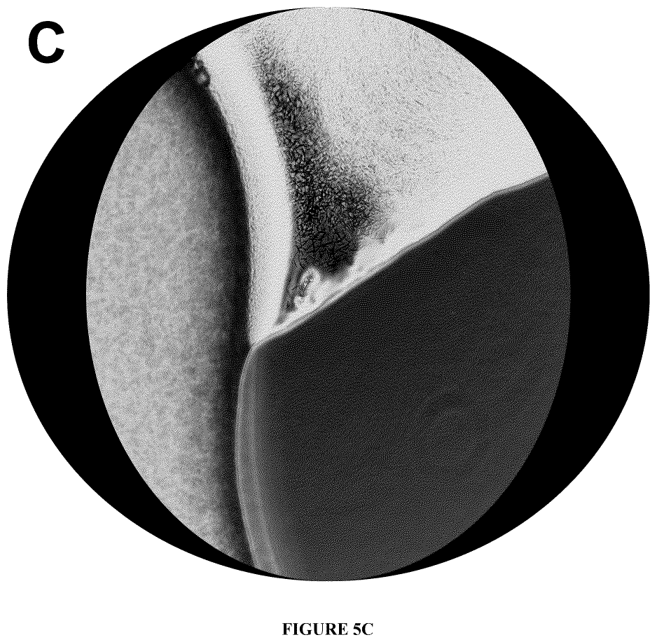

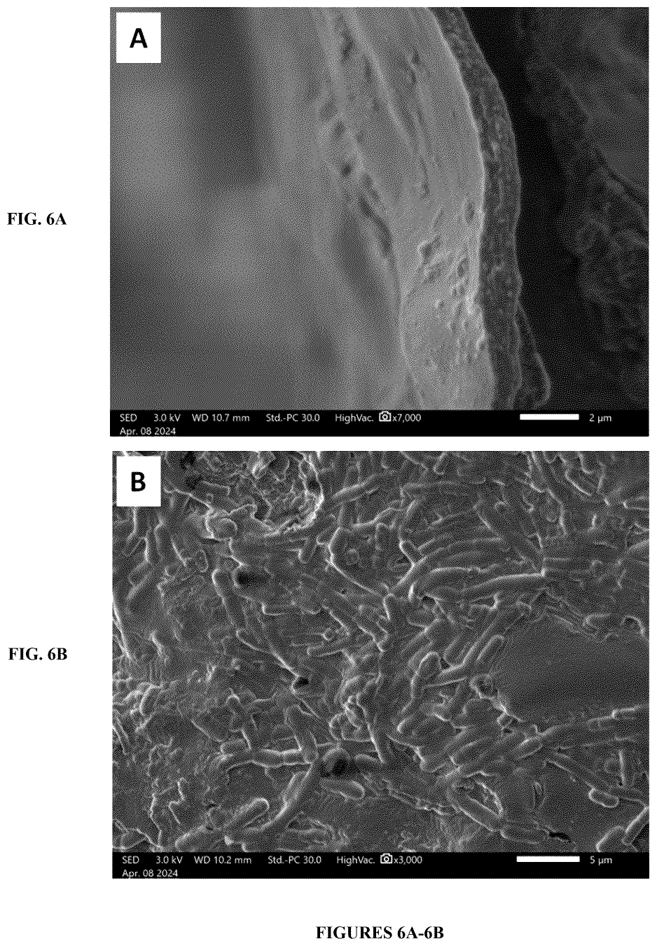

The viability and number of the wildtype LA bacteria growing was analyzed when the bacteria were treated by lyophilization via spiral plating, as shown in FIGS. 5A-5C. The growth of wildtype LA is shown in FIG. 5A, and showed a cell count of approximately 4×10⁴ CFU/mL. Surprisingly, when the wildtype LA was lyophilized followed by viability analysis via spiral plating, more viable bacteria remained, with an approximate cell count of 8×10⁷ CFU/mL (FIG. 5B). Additionally, the lyophilized wildtype LA was imaged using light microscopy to further assess the membrane integrity and viability of the bacteria following lyophilization. Shown in FIG. 5C, the wildtype LA showed high cell counts and encapsulation in a protective medium.

For the recombinant LA stains, a similar procedure was followed. For example, the rLA-DCpep-prM construct was grown to exponential phase, pelleted, washed, and lyophilized using a Labconco benchtop vacuum lyophilizer. The viability of the lyophilized bacteria was verified by spiral plating on MRS agar plates. Several microencapsulation procedures were tested, with one protocol resuspending the lyophilized bacterial in PBS, which was sprayed onto sterilized hulled millet using an airbrush and allowed to dry. The microencapsulation of the rLA was followed by spray-overcoating the treated feed with a 4:1 mixture of polyvinyl alcohol and apple pectin diluted in sterile water (10% w/v) during low-speed agitation in a shaker/mixer until dry. Sample batches of treated feeds were stored at 4° C. for >1 week. Subsequently, 1 g of feed was immersed and agitated in buffer at 37° C. for 1 hr followed by spiral plating of the mixtures on MRS agar and culturing overnight at 37° C.

Fixation of the Wildtype L. acidophilus and Recombinant L. acidophilus to Bird Feed

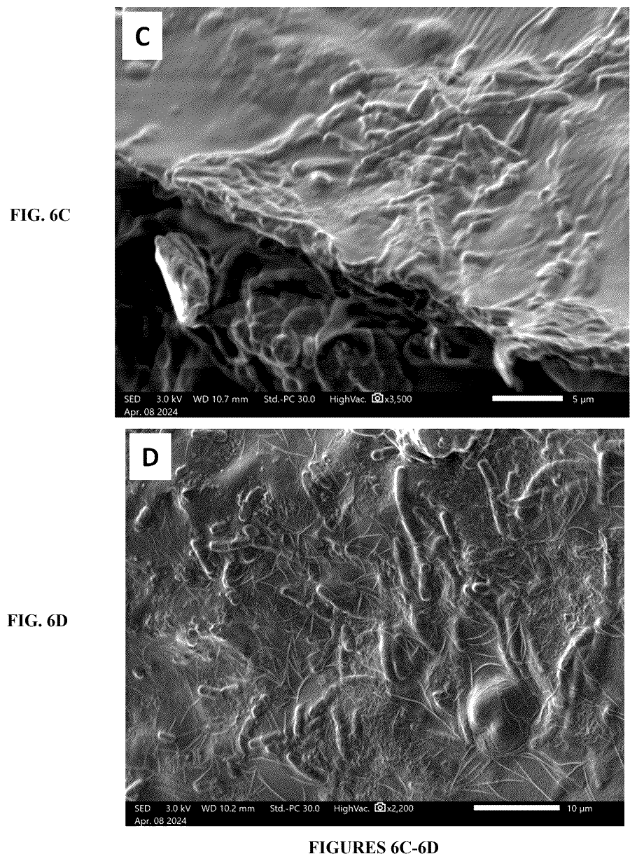

Following successful lyophilization which maintained recombinant protein expression, the lyophilized LA strains were fixed to a variety of tested bird feeds. Various overcoating mixtures were tested including sodium alginate, polyvinyl alcohol, pectin, and agar among others. The lyophilized rLA bacteria was assessed by SEM as shown in FIGS. 6A-6D. Notably, as shown in FIG. 6A and FIG. 6C, the lyophilized rLA bacteria is bound to a seed on the cut seed edge. Similarly, FIG. 6B and FIG. 6D show seeds coated with the microencapsulated rLA overcoated on a seed.

### Example 4

### Immunization of Birds with Recombinant L. acidophilus

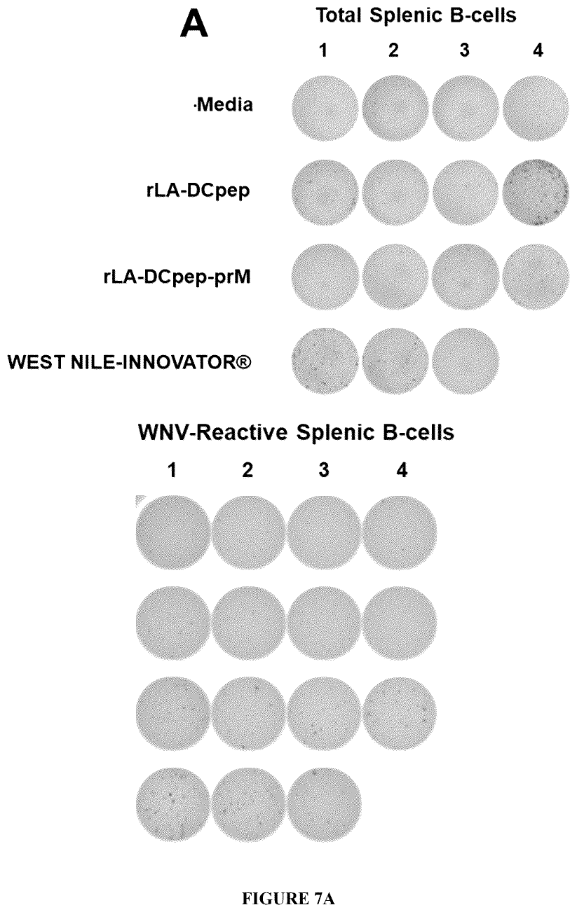

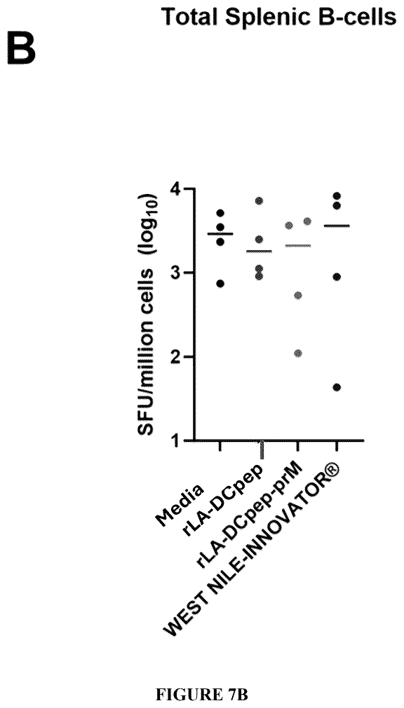

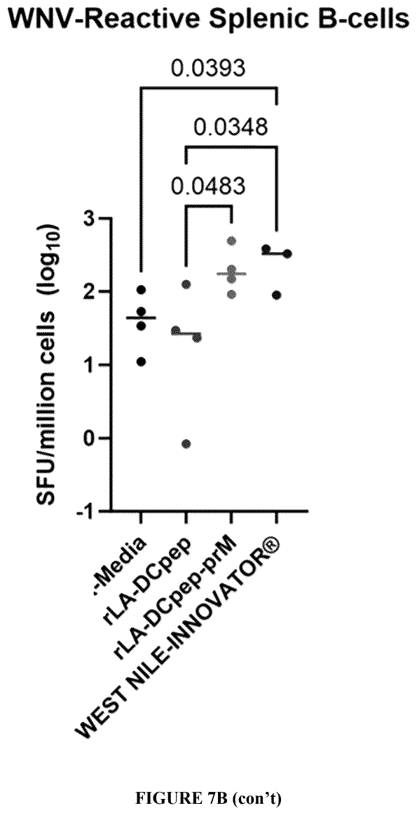

Young chickens have been successfully immunized with the rLA-WNV-prM construct and prM-specific immunity verified by ELISpot which shows WNV-reactive B-cells (FIG. 7A, FIG. 7B). The chickens were immunized by oral gavage with rLA-WNV-prM that was grown in culture flasks, pelleted, washed, counted, and diluted in sterile soy trypsin inhibitor (STI) buffer. The dosing strategy used was inoculation with 5×10⁹ CFU in 200 μL. The dose was administered for 3 consecutive days, every 2 weeks for a total of 3 immunization periods. After immunization, the birds were euthanized and splenocytes harvested and used in ELISpost assays to assess total IgY splenocytes and confirm that prM-specific IgY-secreting B-cells were induced (FIG. 7A, FIG. 7B).

For the ELISPOT, the splenic B cells were stimulated for 3 days ex vivo. FIG. 7A depicts representative wells containing the splenic B-cells and assessment of total IgY and WNV-reactive IgY B cells. The IgY antibodies develop as dark spots within the well, indicating an immunogenic reaction and each spot is equivalent to one B cell. More spots indicate more IgY secreting B cells and in the WNV-reactive wells a stronger immune reaction. rLA media was used as a negative control, and WEST NILE INNOVATOR vaccinated birds were used as a positive control. As can be seen in FIG. 7A, the recombinant LA strain containing the integrated DC peptide only shows increased IgY total B cells compared the media only delivered (FIG. 7A). FIG. 7B is a graph of the specific counts of cells normalized to a million cells. The graphs show that total IgY B cells were normal for all groups and that IgY WNV-reactive B cells were significantly increased in the rLA-DCpep-prM and WEST NILE INNOVATOR groups indicating an increased immune response compared to controls.

Both pre- and post-challenge sera was evaluated by ELISA and plaque reduction neutralization tests (PRNT) to determine antibody titer development. Viral titers in post-challenge sera and post-mortem tissues was quantified by RT-qPCR and statistically analyzed by comparison of means using T-tests. PRNT antibody titers were analyzed by parametric analyses of 50% neutralization values performed from serial dilution assays to generate half-maximal concentration response curves. Post-mortem tissues will be processed for ELISpot assay and antibody production analyzed by ANOVA.

Immunization of Wild Birds Challenged with West Nile Viral Mosquito Infections and Treated with the Immunogenic Bird Feed

Following validation of the rLA constructs and microencapsulation and binding to bird feed, several groups of birds against can be tested with at least two combinations of treated dry bird feed. Groups of 6 birds were be distributed into the following four groups: STI media control, wild-type LA, rLA-DCpep negative control, rLA-DCpep-WNV construct (either premembrane protein (prM), non-structural protein 1 (ns1) or envelope protein (E)). Birds can be orally immunized at days 1-3, 15-17, and 29-31, then challenged on day 45 via intramuscular injection of 105 plaque forming units (PFU) of a WNV strain from Fort Collins, CO grown and titered in Vero cells. Blood can be collected via brachial or jugular venipuncture at a volume no more than 1% of body weight pre-immunization on day 1 as well as days 15, 29, 49, and 52. Birds can be euthanized on day 52 and tissues collected for histologic, RT-qPCR, and ELISpot analysis.

1. A recombinant Lactobacillus acidophilus (rLA) strain comprising one or more antigens integrated into a Lactobacillus acidophilus strain, wherein the one or more antigens comprises a West Nile Virus protein.

2. The rLA strain of claim 1, wherein the West Nile Virus protein is selected from the group consisting of a premembrane (prM) protein, an envelope protein, a non-structural protein 1 (ns1), and any combination thereof.

3. The rLA strain of claim 1, wherein the West Nile Virus protein comprises a sequence having 95% sequence identity to a sequence selected from the group consisting of SEQ ID NO: 7, SEQ ID NO: 8, SEQ ID NO: 9, SEQ ID NO: 10, SEQ ID NO: 11, SEQ ID NO: 12, and any combination thereof.

4. The rLA strain of claim 1, wherein the West Nile Virus protein is encoded by a nucleic acid sequence, wherein the nucleic acid sequence is a codon optimized nucleic acid sequence.

5. The rLA strain of claim 4, wherein the codon optimized nucleic acid sequence comprises a nucleic acid sequence comprising 95% sequence identity to a sequence selected from the group consisting of SEQ ID NO: 2, SEQ ID NO: 4, and SEQ ID NO: 6, and a combination thereof.

6. The rLA strain of claim 1 further comprising an adjuvant.

7. The rLA strain of claim 6, wherein the adjuvant is a dendritic cell (DC) peptide.

8. The rLA strain of claim 6, wherein the adjuvant is encoded by a nucleic acid sequence comprising 95% sequence identity to SEQ ID NO: 13.

9. A lyophilized product comprising the recombinant Lactobacillus acidophilus strain of claim 1.

10. An immunogenic composition comprising a food composition and the lyophilized product of claim 9.

11. The immunogenic composition of claim 10, wherein the lyophilized product is sprayed onto the feed composition.

12. The immunogenic composition of claim 10, wherein the lyophilized product comprises between about 10⁶ to about 10¹² viable microorganisms or between about 10⁶ to about 10¹² microorganisms of the recombinant L. acidophilus strains per gram of the food composition.

13. The immunogenic composition of claim 10, wherein the food composition comprises a bird feed.

14. The immunogenic composition of claim 13, wherein the bird feed is selected from the group consisting of sunflower, safflower, nyjer or thistle, millet, corn, peanuts, milo or sorghum, flax, rapeseed (Brassica napus), canary seed (Phalaris canariensis), fruit, suet, mealworms, and a combination thereof.

15. A method of immunizing an avian, the method comprising a step of administering a recombinant L. acidophilus strain to the avian, wherein the method provides an immunogenic effect in the avian.

16. The method of claim 15, wherein the immunogenic effect comprises stimulation of antigen specific immunity to a West Nile Virus protein.

17. The method of claim 15, wherein the immunogenic effect comprises a reduction in West Nile Virus titer in the avian.

18. The method of claim 15, wherein the recombinant L. acidophilus strain is comprised in a lyophilized product.

19. The method of claim 15, wherein the recombinant L. acidophilus strain is comprised in a feed composition.

20. The method of claim 15, wherein the avian is selected from the group consisting of a native avian, a native-captive avian, an introduced avian, and an exotic-captive avian.


SEQUENCE LISTING (17 sequences)

SEQ ID NO: 1 - DNA, 318 bp, West Nile virus
  source 1..318: mol_type = genomic DNA
atggttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggaggttctacccatcataccactctaccccacaacgcccttaa

SEQ ID NO: 2 - DNA, 276 bp, West Nile virus
  source 1..276: mol_type = genomic DNA
gttaccctctctaacttccaagggaaggtgatgatgacggtaaatgctactgacgtcacagatgtcatcacgattccaacagctgctggaaagaacctatgcattgtcagagcaatggatgtgggatacatgtgcgatgatactatcacttatgaatgcccagtgctgtcggctggtaatgatccagaagacatcgactgttggtgcacaaagtcagcagtctacgtcaggtatggaagatgcaccaagacacgccactcaagacgcagtcggagg

SEQ ID NO: 3 - DNA, 1533 bp, West Nile virus
  source 1..1533: mol_type = genomic DNA
atgtttaactgtttgggtatgtctaaccgtgacttccttgaaggagtaagcggtgccacatgggtcgatcttgttcttgagggtgattcttgcgttaccattatgagtaaggacaaaccaacaatcgacgtcaaaatgatgaatatggaggctgccaacttagctgacgtccgctcatactgttatttggcttctgtcagcgatctttctacccgtgccgcatgtcctacaatgggtgaggctcacaatgagaagcgagcagacccagcttttgtctgcaagcagggcgtagttgatcgaggctggggtaatggatgtggattatttggcaaaggatctattgacacctgcgcaaagttcgcttgcactaccaaagccacaggttggatcattcaaaaggagaatatcaaatacgaggtagccattttcgtacacggaccaacaactgttgagtcacatggcaaaatcggtgcaactcaagcaggcagattttctattaccccttcagcaccatcatacactttaaagcttggagagtatggcgaggttacagttgattgcgaaccaagaagcggaatcgacacctcagcttactacgtcatgtctgtaggcgagaaaagttttttagtacacagagagtggttcatggatcttaatttgccttggagttctgccggctctaccacatggcgcaatcgcgaaacccttatggagttcgaggagcctcatgccaccaagcaatctgttgttgccttgggtagtcaagaaggtgccttgcatcaggcacttgctggcgctattcctgttgagtttagttctaacaccgtcaaattgacctcaggtcacttgaaatgtcgagttaagatggaaaagcttcaacttaagggtaccacttatggcgtttgctctaaggcctttaagttcgctcgtactcctgccgataccggacatggtaccgtcgttcttgagcttcagtacactggaaccgacggtccttgtaaagttcctattagctctgtcgcctctttaaacgatttgacccctgtcggacgtttggtaacagtaaatccattcgtcagtgtagcaaccgcaaactcaaaagtattaattgagttagagcctccatttggcgactcttatatcgttgttggacgaggcgaacagcaaattaatcatcattggcacaaatcaggaagcagcatcggcaaggcatttactactacattgcgtggcgcacaaagattggctgcacttggagataccgcctgggatttcggcagcgttggcggagttttcacctctgtaggcaaagcaatccaccaagtctttggtggcgccttccgcagcttattcggtggaatgtcatggattacacagggattacttggtgcccttttgctttggatgggtatcaacgctagagaccgcagcattgcaatgacctttttagctgtcggtggtgtattgctttttctttcagtcaacgtacacgctttctacccatcataccactctaccccacaacgcccttaa

SEQ ID NO: 4 - DNA, 1503 bp, West Nile virus
  source 1..1503: mol_type = genomic DNA
tttaactgtcttggcatgtcaaaccgcgatttccttgagggagtaagcggcgccacatgggtcgacttggtccttgaaggcgattcttgcgtaaccatcatgtctaaagataagccaacaatcgatgtaaagatgatgaacatggaggccgccaatttagctgaggtacgcagttactgttaccttgctactgtaagcgatttgtcaactaaggccgcatgtccaacaatgggagaagcacataacgataagcgcgccgaccctgctttcgtatgccgacagggagttgttgatagaggctggggaaacggatgtggtttatttggaaaaggttcaatcgatacctgtgctaagtttgcatgtagcacaaaagccatcggtagaactattttgaaagaaaacatcaaatacgaggttgccatctttgtacatggaccaaccactgttgagagccacggtaactattcaacacaggttggcgctacacaggccggacgtttcagcatcacaccagcagcaccatcttatactttaaaattgggagagtatggcgaagtcaccgtagactgtgagccacgatctggaattgataccaatgcctattacgttatgactgttggtaccaaaacttttttagtacatcgcgaatggtttatggacttaaacttaccttggtcaagcgccggcagtaccgtttggcgaaatcgcgagaccttaatggagttcgaagagccacacgccactaagcagagcgtcattgcattgggctctcaggagggtgcattacatcaggcacttgcaggagccatcccagtagaattctcaagtaatacagttaagttaacaagtggacatcttaaatgtcgcgtcaaaatggagaaattgcagttgaagggtacaacttatggcgtctgttctaaggctttcaaatttttaggcaccccagccgacacaggtcacggcactgtcgttcttgaattgcaatatacaggaacagatggaccttgtaaagtacctatcagctcagtcgccagtcttaatgaccttacacctgttggccgacttgttactgttaacccttttgtctctgttgcaaccgctaacgctaaggtattaatcgaacttgagccaccattcggagactcttatatcgtagtcggtcgaggagagcagcaaatcaaccaccattggcacaaatctggaagcagcatcggtaaggcttttaccactacattaaagggagcccagcgacttgcagcccttggcgacacagcttgggactttggctctgttggaggagtttttacttctgttggaaaagcagtccaccaggttttcggcggagcctttagaagtttatttggaggtatgagttggatcacccaaggattacttggagctttgcttctttggatgggtattaatgcccgagatcgtagtattgccttaacttttcttgcagtaggtggagttttattatttttaagtgttaacgtccatgca

SEQ ID NO: 5 - DNA, 1098 bp, West Nile virus
  source 1..1098: mol_type = genomic DNA
atggacaccggttgcgccatcgacattagtcgacaagaattacgttgcggttcaggagtattcatccataacgacgtagaagcttggatggacagatacaagtactatccagagaccccacagggacttgcaaagatcatccaaaaggcacataaggaaggagtctgcggtcttagatctgtctctcgattggagcatcagatgtgggaggccgtaaaagacgagcttaacaccttgcttaaggagaatggcgtagacttatctgttgttgttgaaaaacaagaaggcatgtataagagtgctccaaagcgtcttaccgcaacaactgaaaagttggagatcggatggaaagcttggggaaaatcaattttatttgcaccagaattggctaacaacacctttgtcgtagacggtcctgagactaaggaatgccctacccagaacagagcctggaattcattagaagtcgaggacttcggctttggccttacctctacccgaatgttcttaaaagttagagagagtaacaccacagaatgcgacagcaagatcattggcacagccgtcaagaacaatttagctattcacagcgatttgagctattggatcgaatctcgccttaacgacacatggaaattagaacgcgcagtattaggagaagtaaagtcttgcacatggcctgaaacacacaccttatggggagatggcattcttgagagtgaccttattattcctgtaaccttagcaggaccacgatctaatcataatcgccgcccaggatacaagactcagaaccagggtccttgggatgaaggacgtgttgaaatcgactttgattattgtcctggcaccacagttacacttagtgagtcttgtggtcatcgaggtcctgctacccgcactactacagaaagtggcaaattgattacagactggtgctgtcgatcatgtacattgcctccattgcgttaccagaccgatagcggctgttggtatggtatggagatccgtccacaacgtcacgatgagaaaactcttgtacaatctcaagtcaatgctttctacccatcataccactctaccccacaacgcccttaa

SEQ ID NO: 6 - DNA, 1056 bp, West Nile virus
  source 1..1056: mol_type = genomic DNA
gacaccggttgcgccatcgacattagtcgacaagaattacgttgcggttcaggagtattcatccataacgacgtagaagcttggatggacagatacaagtactatccagagaccccacagggacttgcaaagatcatccaaaaggcacataaggaaggagtctgcggtcttagatctgtctctcgattggagcatcagatgtgggaggccgtaaaagacgagcttaacaccttgcttaaggagaatggcgtagacttatctgttgttgttgaaaaacaagaaggcatgtataagagtgctccaaagcgtcttaccgcaacaactgaaaagttggagatcggatggaaagcttggggaaaatcaattttatttgcaccagaattggctaacaacacctttgtcgtagacggtcctgagactaaggaatgccctacccagaacagagcctggaattcattagaagtcgaggacttcggctttggccttacctctacccgaatgttcttaaaagttagagagagtaacaccacagaatgcgacagcaagatcattggcacagccgtcaagaacaatttagctattcacagcgatttgagctattggatcgaatctcgccttaacgacacatggaaattagaacgcgcagtattaggagaagtaaagtcttgcacatggcctgaaacacacaccttatggggagatggcattcttgagagtgaccttattattcctgtaaccttagcaggaccacgatctaatcataatcgccgcccaggatacaagactcagaaccagggtccttgggatgaaggacgtgttgaaatcgactttgattattgtcctggcaccacagttacacttagtgagtcttgtggtcatcgaggtcctgctacccgcactactacagaaagtggcaaattgattacagactggtgctgtcgatcatgtacattgcctccattgcgttaccagaccgatagcggctgttggtatggtatggagatccgtccacaacgtcacgatgagaaaactcttgtacaatctcaagtcaatgct

SEQ ID NO: 7 - protein, 92 aa, West Nile virus
VTLSNFQGKVMMTVNATDVTDVITIPTAAGKNLCIVRAMDVGYMCDDTITYECPVLSAGNDPEDIDCWCTKSAVYVRYGRCTKTRHSRRSRR

SEQ ID NO: 8 - protein, 501 aa, West Nile virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 9 - protein, 401 aa, West Nile virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQVGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSG

SEQ ID NO: 10 - protein, 352 aa, West Nile virus
DTGCAIDISRQELRCGSGVFIHNDVEAWMDRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLKVRESNTTECDSKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTESGKLITDWCCRSCTLPPLRYQTDSGCWYGMEIRPQRHDEKTLVQSQVNA

SEQ ID NO: 11 - protein, 141 aa, West Nile virus
DRYKYYPETPQGLAKIIQKAHKEGVCGLRSVSRLEHQMWEAVKDELNTLLKENGVDLSVVVEKQEGMYKSAPKRLTATTEKLEIGWKAWGKSILFAPELANNTFVVDGPETKECPTQNRAWNSLEVEDFGFGLTSTRMFLK

SEQ ID NO: 12 - protein, 123 aa, West Nile virus
SKIIGTAVKNNLAIHSDLSYWIESRLNDTWKLERAVLGEVKSCTWPETHTLWGDGILESDLIIPVTLAGPRSNHNRRPGYKTQNQGPWDEGRVEIDFDYCPGTTVTLSESCGHRGPATRTTTE

SEQ ID NO: 13 - DNA, 36 bp, synthetic construct
  source 1..36: mol_type = other DNA
ttctacccatcataccactctaccccacaacgccct

SEQ ID NO: 14 - protein, 12 aa, synthetic construct
FYPSYHSTPQRP

SEQ ID NO: 15 - DNA, 4114 bp, synthetic construct
  source 1..4114: mol_type = other DNA
actcggcacttaatggggggtcgtagtacggaagcaaaattcgcttcctttccccccatttttttccaaattccaaatttttttcaaaaattttccagcgctaccgctcggcaaaattgcaagcaatttttaaaatcaaacccatgagggaatttcattccctcatactcccttgagcctcctccaaccgaaatagaagggcgctgcgcttattatttcattcagtcatcggctttcataatctaacagacaacatcttcgctgcaaagccacgctacgctcaagggcttttacgctacgataacgcctgttttaacgattatgccgataactaaacgaaataaacgctaaaacgtctcagaaacgattttgagacgttttaataaaaaatcgctagtccgaggcctcgacccgattcacaaaaaataggcacacgaaaaacaagttaagggatgcagtttatgcatcccttaacttacttattaaataatttatagctattgaaaagagataagaattgttcaaagctaatattgtttaaatcgtcaattcctgcatgttttaaggaattgttaaattgattttttgtaaatattttcttgtattctttgttaacccatttcataacgaaataattatacttttgtttatctttgtgtgatattcttgatttttttctacttaatctgataagtgagctattcactttaggtttaggatgaaaatattctcttggaaccatacttaatatagaaatatcaacttctgccattaaaagtaatgccaatgagcgttttgtatttaataatcttttagcaaacccgtattccacgattaaataaatctcattagctatactatcaaaaacaattttgcgtattatatccgtacttatgttataaggtatattaccatatattttataggattggtttttaggaaatttaaactgcaatatatccttgtttaaaacttggaaattatcgtgatcaacaagtttattttctgtagttttgcataatttatggtctatttcaatggcagttacgaaattacacctctttactaattcaagggtaaaatggccttttcctgagccgatttcaaagatattatcatgttcatttaatcttatatttgtcattattttatctatattatgttttgaagtaataaagttttgactgtgttttatatttttctcgttcattataaccctctttaatttggttatatgaattttgcttattaacgattcattataaccttacttagtaccgaagagtctgtcaccggcatctcccaaaccagggaaaatgtaaccattgtccagcaatttatcatcttctgatgcagcataaatatcaacatcaggattttcttcttggactgccttaataccttctggagctgctactaaaactgccaatttaatatccttaacaccacgcttctttaatgcagcgatagcgtcattagcagaaccaccagtagcaagcattggatcaacaactaaacattcacgttcagcaatatcctttggagctttaaagaaatattcatgcggcttcaaagtttcttcatcacgatacataccaataacagcaatcttagctgatggtaccatctcaagaacaccgttaagcattcccatacctgcacgcaaaatcggcacaattgtcaatttcttgccggcgatttctttttggactgtcttacccattggtgtttcaatttcaacatcttcaagtggtaaatctctagtaatttcataggtcattaatccaccgatttcaccaacaattctacgaaattcgtttgaacctgtgtccttgcgacgaataattgttaatttgtgctgaatcaaagggtgatccaaaactacaaactttcccatatatgcctcctattaacaaataacttaattcattaaatattttaatcaaaaacgcaaagggtttaaagccctttgcgttttattttttatttattcaaatgatggccgccggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcgatagcgattgtattagtaactaatttattagctggatgtagtcaaatccattttggcaaagatgcaatcacaattggtgatgagcaaaaaaatcttttaaagaagaataaaaagaaaaaagtagtacatcctaaaattgaaaagaaaaaaacaaaagtaaaaagtgtttggaacaaaacaaagtatcaggaattacaaaagattgtaaataattggggtagggtaaaaaaacaacattatcatttttatgatggcgtccattctttaaaaataaaagcaggagttacatatccaaaagcttttgatcaaaatggtttcgttttaaataaagaaaaaatcaaaatagggtggagtccagaaggtaaaaatacgtatcaatacaatgttgtagcaattgccaatgataattttgaaacatggcacaatacatatttgttttgtttaagaaagaataaaccaattattttattagatcagtcaaaacgaaataagcttgtattagttaagaaagtgaataatcctactcttaataaagcctttaaacaaattttagaagataaacaaaaaagcatgtagaaaattaatctacatgctttttgttgaaattgtactcggtgcggccgcattaatcgtggtcaacatcatctgggaagaatgcaagacgttcgcccttgccaagttcttcttcgatttcaagtaagcggttgtacttttcaacacgttctgaacgagctggagcaccggtcttaagttgaccaccgtttatagcaactgccaagtcagcgatgaatgtgtcaccagtttcacctgaacggtgtgaaatcatagtgttgtaaccattcttacgtgacatacggatagtttcaagagtttcagtaacagtaccaatttggttcaacttgattaatgaagcgttacctgcaccttccttgatagccttcttcaaaagtgctgggttagtacaaatgaagtcgtcaagaacgatttggatacggtccttgtgagttgcagtaaacttaaccataccttcaacatcgttttcgtcgtatgggtcttccattgacattaattctgggaattcatccaaaagcttgtcgtagtatgcagctaattcatcatcagtaagaaccttaccttcaaggtggtacttaccatcttccttgttgtagaagtatgaagcagcacagtcacatgcgataccgatatcttcacctggcttgtaaccagccttgatgattgattcgtgtaaagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcagctagttctagaactagcgattctgaaatcaccatttaaaaaactccaatcaaataattttataaagttagtgtatcactttgtaatcataaaaacaacaataaagctacttaaatatagatttataaaaaacgttggcgaaaacgttggcgattcgttggcgattgaaaaaccccttaaacccttgagccagttgggatagagcgtttttggcacaaaaattggc

SEQ ID NO: 16 - DNA, 3024 bp, synthetic construct
  source 1..3024: mol_type = other DNA
gactgctagcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgaaaaaaagagttatgatttctctgacaaaagagcaagataaaaaattaacagatatggcgaaacaaaaaggtttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagccccctgacgaaagtcgaagggggtttttattttggtttgatgttgcgattaatagcaatacaattgcaataaacaaaatgatcttccttcaggttatgaccatctgtgccagttcgtaatgtctggtcaactttccgactctgagaaacttctggaatcgctagagaatttctggaatgggattcaggagtggacagaacgacacggatatatagtggatgtgtcaaaacgcataccattttgaacgatgacctctaataattgttaatcatgttggttacgtatttattaacttctcctagtattagtaattatcatggctgtcatggcgcattaacggaataaagggtgtgcttaaatcgggccattttgcgtaataagaaaaaggattaattatgagcgaattgaattaataataaggtaatagatttacattagaaaatgaaaggggattttatgcgtgagaatgttacagtctatcccggcaatagttacccttattatcaagataagaaagaaaaggatttttcgctacgctcaaatcctttaaaaaaacacaaaagaccacattttttaatgtggtctttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataatatgagataatgccgactgtactttttacagtcggttttctaatgtcacttctagatctg

SEQ ID NO: 17 - DNA, 5009 bp, synthetic construct
  source 1..5009: mol_type = other DNA
agcttcatctgaggataaagttgtttgataaatgctcaactttaagtaattttaaggagctaactaactgtgggggatgaaataaagccaatagaaaaagcgaacctaataagattaatctttaggaaaatcgaataaaaatattacttttttgatatgttttgtcatagtttcgtaaaatttagtaaagattacgagcgataaatagagaacttaatcttgtctttttcttgctatagctaggtttagcacattttacaattttaaagtgcttgtaatgcttgtgggggtaagcggtaggtgaaatattacaaatagtatttttcggtcattttaacttgctatttcttgaagaggttagtacaatatgaatcgtggtaagtaataggacgtgcttcaggcgtgttgcctgtacgcatgctgattcttcagcaagactactacctcatgagagttatagactcatggatcttgctttgaagggttttgtacattataggctcctatcacatgctgaacctatggcctattacatttttttatatttcaaggaggaaaagaccacatgaagaaaaatttaagaatcgttagcgctgctgctgctgctttacttgctgttgctccagttgctgcttctgctgtatctactgttagcgctgctactactattaacgcaagttcatcagcaatcaataccaacactaatgctaagtacgatgttgatgtaactcctagtgtttctgcagttgctgcaaatactgctaacaacactccagctattgccggtaaccttactggtactatttcagcaagttacaatggtaagacttatactgctaacttaaaggcagatactgaaaatgccactattactgctgctggtagcactactgccgttaaacctgctgaattagctgcaggtgtggcttacactgtaactgttaacgatgtttcatttaacttcggttcagaaaatgcaggtaagactgttacccttggttcagctaactcaaatgtaaaattcaccggtacaaacagtgataatcaaactgaaactaatgtttctactttgaaagttaagttagaccaaaacggtgttgcttcacttactaatgtttcaattgcaaacgtatacgcaattaacactactgataacagtaacgtaaacttctacgacgtaactagtggtgctactgtaactaacggtgccgtttcagttaatgctgataaccaaggtcaagttaatgttgcaaacgtagttgcagcaattaattcaaaatactttgcagcacaatacgcagataagaagttaaatactcgtactgctaatactgaagatgctattaaggcagccttaaaggaccaaaagattgatgtaaactcagtaggttacttcaaagcacctcatactttcactgttaacgttaaagcaacttcaaatactaatggtaagtcagctactttgccagtagttgttactgttcctaatgttgctgagccaactgtagccagcgtaagcaagagaattatgcacaacgcatactactacgacaaggacgctaagcgtgttggtactgacagcgttaagcgttacaactcagtaagcgtattgccaaacactactactatcaacggtaagacttactaccaagtagttgaaaacggtaaggctgttgacaagtacatcaacgctgcaaacatcgatggtactaagcgtactttgaagcacaacgcttacgtttacgcatcatcaaagaagcgtgctaacaaggttgtattgaagaagggtgaagttgtaactacttacggtgcttcatacacattcaagaacggccaaaagtactacaagatcggtgacaacactgacaagacttacgttaaggttgcaaactttagataataagtcgtagcactaacgctaacaaaatgaaaaaggcagagcgaaagctctgtctttttttgtgttttaaatttttatttcacttctaaacatacagttctaataggatcttctgatacttcgattcggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccggcggccatcatttgaataaataaaaaataaaacgcaaagggctttaaaccctttgcgtttttgattaaaatatttaatgaattaagttatttgttaataggaggcatatatgggaaagtttgtagttttggatcaccctttgattcagcacaaattaacaattattcgtcgcaaggacacaggttcaaacgaatttcgtagaattgttggtgaaatcggtggattaatgacctatgaaattactagagatttaccacttgaagatgttgaaattgaaacaccaatgggtaagacagtccaaaaagaaatcgccggcaagaaattgacaattgtgccgattttgcgtgcaggtatgggaatgcttaacggtgttcttgagatggtaccatcagctaagattgctgttattggtatgtatcgtgatgaagaaactttgaagccgcatgaatatttctttaaagctccaaaggatattgctgaacgtgaatgtttagttgttgatccaatgcttgctactggtggttctgctaatgacgctatcgctgcattaaagaagcgtggtgttaaggatattaaattggcagttttagtagcagctccagaaggtattaaggcagtccaagaagaaaatcctgatgttgatatttatgctgcatcagaagatgataaattgctggacaatggttacattttccctggtttgggagatgccggtgacagactcttcggtactaagtaaggttataatgaatcgttaataagcaaaattcatataaccaaattaaagagggttataatgaacgagaaaaatataaaacacagtcaaaactttattacttcaaaacataatatagataaaataatgacaaatataagattaaatgaacatgataatatctttgaaatcggctcaggaaaaggccattttacccttgaattagtaaagaggtgtaatttcgtaactgccattgaaatagaccataaattatgcaaaactacagaaaataaacttgttgatcacgataatttccaagttttaaacaaggatatattgcagtttaaatttcctaaaaaccaatcctataaaatatatggtaatataccttataacataagtacggatataatacgcaaaattgtttttgatagtatagctaatgagatttatttaatcgtggaatacgggtttgctaaaagattattaaatacaaaacgctcattggcattacttttaatggcagaagttgatatttctatattaagtatggttccaagagaatattttcatcctaaacctaaagtgaatagctcacttatcagattaagtagaaaaaaatcaagaatatcacacaaagataaacaaaagtataattatttcgttatgaaatgggttaacaaagaatacaagaaaatatttacaaaaaatcaatttaacaattccttaaaacatgcaggaattgacgatttaaacaatattagctttgaacaattcttatctcttttcaatagctataaattatttaataagtaagttaagggatgcataaactgcatcccttaacttgtttttcgtgtgcctattttttgtgaatcgggtcgaggcctcggactagcgattttttattaaaacgtctcaaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgcccttctatttcggttggaggaggctcaagggagtatgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttaaggggtttttcaatcgccaacgaatcgccaacgttttcgccaacgttttttataaatctatatttaagtagctttattgttgtttttatgattacaaagtgatacactaactttataaaattatttgattggagttttttaaatggtgatttcagaatcgctagttctagaactagctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcca